(19) United States
(12) Patent Application Publication
(10) Pub. No.: US 2013/0130278 A1
(43) Pub. Date: May 23, 2013

(54) DETECTION OF RISK FOR PREGNANCY-RELATED MEDICAL CONDITIONS

(71) Applicant: Ottawa Hospital Research Institute, Ottawa (CA)

(72) Inventors: Andree GRUSLIN, Cantley (CA); Qing QIU, Ottawa (CA)

(73) Assignee: OTTAWA HOSPITAL RESEARCH INSTITUTE, Ottawa (CA)

(21) Appl. No.: 13/680,645

(22) Filed: Nov. 19, 2012

Related U.S. Application Data
(60) Provisional application No. 61/562,620, filed on Nov. 22, 2011.

(51) Int. Cl. G01N 33/68

(52) U.S. Cl. CPC G01N 33/689
USPC 435/7.5; 435/7.1; 435/7.92

(57) ABSTRACT

Generally, there is provided a method of determining risk of determining intrauterine growth restriction (IUGR)/fetal growth restriction (FGR). The method comprises obtaining a sample from a pregnant woman, taking a measurement indicative of IGFBP-4 level in the sample, and determining that increased of IUGR/FGR exists if the IGFBP-4 level is elevated. The sample may be taken during the first trimester of pregnancy, and may comprise maternal serum. The measurement may be one of IGFBP-4 protein which may be made using an immunoassay, such as a Western blot or an enzyme-linked immunosorbent assay (ELISA).

### RELATED APPLICATIONS

This application claims the benefit of priority of U.S. Provisional Application No. 61/562,620 filed on Nov. 22, 2011, which is incorporated herein by reference in its entirety.

### FIELD

The present disclosure relates generally to detection of medical conditions. More particularly, the present disclosure relates to detection of risk for pregnancy-related medical conditions.

### BACKGROUND

Premature births and undesirable prenatal and perinatal conditions can lead to serious complications for the mother, the delivery of a baby, the mortality of the baby, and can also lead to a higher risk of the baby of developing a long-term handicap, such as developmental delay, cerebral palsy, blindness, deafness, and chronic lung disease.

Intrauterine growth restriction (IUGR), also known as fetal growth restriction (FGR) is a leading cause of perinatal mortality and morbidity in the developed world. It is usually diagnosed by ultrasound (umbilical artery and ductus veniosus Doppler), and the only clinical management strategy for IUGR is delivery. However, it is currently impossible to accurately predict, prevent, or treat IUGR, and there is no testing available for IUGR in early, or first trimester, pregnancy.

This condition is characterized by abnormalities in early implantation of the placenta in the maternal decidua (55-57).

The rapid identification of patients at risk of such conditions is highly desirable. Conventional methods of identifying these conditions individually or concurrently are not necessarily objective, sensitive, or specific (45). Extant analytes have inadequately low predictive value for IUGR, and none of these would be useful in standalone tests which would be early, reliable or strong enough to support the prediction of IUGR.

For example, Brask et al. (47) studied the association between serum YKL-40 and uterine artery Doppler flow measured at week 28 and the development of pre-eclampsia and (IUGR) later in pregnancy. YKL-40 was evaluated to be a useful as a biomarker for preeclampsia, but not for IUGR. U.S. Pat. No. 7,955,805 B2 uses insulin-like growth factor binding protein-5 (IGFBP5) measured from the maternal serum in the first trimester of pregnancy as an indicator of pre-eclampsia or eclampsia, or as a predisposition thereto, but not as an indicator of IUGR. Chaftez et al. (47) screened maternal PP13 levels in the first trimester and correlated it to the incidence of pre-eclampsia and IUGR, but found that it was a promising diagnostic tool only for the prediction of pre-eclampsia with high sensitivity and specificity. There was no correlation between IUGR and PP13. Barkehall-Thomas et al. (48) considered maternal serum activin A as an indicator of IUGR, but the levels of detection were unlikely to be sufficient for clinical utility. Other predictors of placental insufficiency and IUGR have included maternal PAPP-A or free βHCG, α-fetoprotein (AFP), early fetal growth and abnormal uterine artery Doppler (16; 27-34). However, these analytes have inadequately low predictive value for IUGR, and none of these would be useful in standalone tests which would be early, reliable or strong enough to support the prediction of IUGR; or IUGR and pre-eclampsia.

The study of undesirable prenatal and perinatal conditions focuses on the interaction of complex systems that cells use to interact with their physiologic environment. Insulin-like growth factors and insulin growth factor binding proteins are part of these complex systems. Of particular interest in pregnancy, insulin-like growth factor II (IGF-II) is expressed in early pregnancy by maternal, fetal and placental tissues. It is believed that insufficient IGF-II results in limited placenta growth, thereby restricting nutrient transfer to the developing fetus and leading to fetal growth restriction (3; 4). However, the specific role of dysregulated IGF signaling in the development of IUGR remains poorly understood. The mechanism of action of insulin-like growth factor (IGF) and its bioavailiability are regulated by six high affinity binding proteins (IGFBP1 to 6). Of these, insulin-like growth factor-binding protein 4 (IGFBP-4) along with IGFBP1 are the most abundant IGFBPs in the placental bed (5).

Current in vitro data suggest that IGFBP-4 acts as an inhibitor of IGF activity. IGFBP-4 activity, in turn, can be directly regulated through the proteolytic activity of Pregnancy-Associated Protein A (PAPP-A) (6). Through PAPP-A activity, IGFBP-4 degradation results in a release of IGFBP-4-bound IGF-II which allows a local increase in IGF-II bioavailability (7). In the context of pregnancy, IGFPB4 is thought to play an important role in IGF-II signaling within the feto-placental unit (7). Mice with homozygous deletion of PAPP-A have increased concentrations of circulating IGFBP-4 and a 34% reduction in fetal weight (8). Interestingly, knocking out IGFBP-4 in PAPP-A null mice results in only a 7% reduction of fetal weight compared with WT littermate (9). This attenuation of fetal growth restriction in the double knockout mice compared with the single PAPP-A knockout further supports the possibility that growth restriction in PAPP-A null mice may be through an increase in IGFBP-4-mediated action.

However, while reduced levels of circulating PAPP-A has been shown to be a useful first trimester marker for Down's syndrome (10-12) and have also been correlated with an increased risk of low birth weight and IUGR, PAPP-A has not been recognized on its own as a clinically useful predictor of IUGR (13-17). The relationship between PAPP-A and circulating levels of IGFBP-4 in the maternal serum, and whether this relationship is a cause or consequence of aberrant placental development in complicated pregnancies, is not clear.

IGFBP-4 levels have been studied in fetuses in induced animal models of IUGR, wherein elevated levels of IGFBP-4 were associated with IUGR in induced guinea pig, rat, and sheep models (51-53). However, it is well-known that circulating fetal protein and proteins expressed by the placenta are not reliably associated with circulating maternal protein levels. Further the induced nature of these models renders them of questionable relevance to IUGR in humans.

In summary, the underlying mechanisms of IUGR in humans is unclear. There is currently no test that predicts IUGR in humans early in pregnancy.

It would be desirable to provide a simple test that predicts IUGR early in pregnancy and/or with greater accuracy than forgoing methodologies.

### SUMMARY

It is an object of the present disclosure to obviate or mitigate at least one disadvantage of previous approaches.

In a first aspect, the present disclosure provides a method of determining risk of intrauterine growth restriction (IUGR) during pregnancy comprising the steps of: obtaining a sample from a pregnant woman; taking a measurement indicative of an IGFBP-4 level in the sample; and determining that an increased risk of IUGR exists if the IGFBP-4 level is elevated.

In one aspect there is provided a kit for determining a risk of intrauterine growth restriction (IUGR) during pregnancy from a sample obtained from a pregnant woman comprising: reagents for taking a measurement indicative of an IGFBP-4 level in said sample; and instructions for use, wherein an increased risk of IUGR is determined if the level of IGFBP-4 is elevated.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts representative immunohistochemistry (IHC) showing PAPP-A and IGFBP-4 in extravillous trophoblast and decidual cells at the maternal-fetal interface tissue at 13 weeks of gestational age. Panel a depicts staining for cytokeratin as a extravillous trophoblast marker. Panel b depicts staining for vimentin as a decidual cell marker. IHC for PAPP-A and IGFBP-4 was also conducted on adjacent sections to identify the expression of PAPP-A and IGFBP-4 in specific cell types. Panel c depicts staining for PAPP-A. Panel d depicts staining for IGFBP-4. The circled area highlights cytokeratin positive cells (extravillous trophoblasts) and a few vimentin positive cells (decidual cells), while boxed area highlights decidual cells (vimentin positive) intensively distributed and a few extravillous trophoblasts scattered in this area.

FIG. 2 depicts the results of a Western blot of aliquots of 7.5 μl diluted serum samples (1:20) with 1× loading buffer (as well as protein ladder and internal control) which had been subjected to 6 SDS-PAGEs; and indicates that there was equal sample loading and consistency of protein transfer.

FIG. 3 depicts immunostaining results for PAPP-A and IGFBP-4 expression. Panel a depicts a section containing placental villi at 13 weeks gestational age that is immunostained for PAPP-A. Panel b showing staining (of a destained section from panel a) with cytokeratin as a cytotrophoblast trophoblast marker. Panel c shows immunostaining of IGFBP-4 conducted in adjacent serial section.

FIG. 4 depicts data suggesting that IGFBP-4 is predominantly expressed by HTR8/SVneo cells and can complex with IGF-II. IGFBP-4 is predominately secreted by human extravillous trophoblasts and forms a complex with IGF-II. Panel a shows that three IGFBP's were identified in the conditioned media of HTR8/SVneo cells, with confirmed identify of IGFBP2, IGFBP3 and IGFBP-4 through WB. Panel b depicts WB analysis for IGF-II and IGFBP-4 conducted on conditioned media with or without cross-linking conditions. Panel c depicts IGFBP-4 WB analysis showed IGFBP-4 decrease in cross-linked samples.

FIG. 5 depicts data indicating that IGFBP-4 is increased in the circulation of pregnant women in early gestation, while circulating PAPP-A levels increase across gestation. Panel a shows WB analysis of circulating IGFBP-4 in 1st trimester (7-10 weeks) serum samples compared to samples from non-pregnant and pregnant women in 2nd (17-21 weeks) and 3rd trimester (37-40 weeks). Panel b shows parallel WB analysis of circulating PAPP-A.

FIG. 6 depicts multi-strip WB and WLB analysis showed no correlation between circulating IGF-I, IGF-II in early gestation in with women destined to deliver growth restricted infants. Panel a depicts the results multi-strip WB analysis for IGF-I in maternal serum samples. Panel b depicts subsequent detection of IGF-II.

FIG. 7 shows that circulating IGFBP-4 was elevated in early gestation in women destined to deliver a FGR infant. Panel a shows multi-strip WB analysis of in serum samples of women who went on to develop FGR. Samples with elevated IGFBP-4 are annotated on the blot with a white * (in the control group) and black * (in the FGR group). Panel b shows parallel analysis of PAPP-A expression. Panel c shows quantification of WB data from panel a. Panel d shows quantification of WB data from panel b. Panel e shows a plot of IGFBP-4 and PAPP-A concentrations in all samples. The data are represented with median; 25th-75th percentiles.

### DETAILED DESCRIPTION

Generally, there is provided a method of determining risk of determining intrauterine growth restriction (IUGR)/fetal growth restriction (FGR). The method comprises obtaining a sample from a pregnant woman, taking a measurement indicative of IGFBP-4 level in the sample, and determining that increased of IUGR/FGR exists if the IGFBP-4 level is elevated. The sample may be taken during the first trimester of pregnancy, and may comprise maternal serum. The measurement may be one of IGFBP-4 protein which may be made using an immunoassay, such as a Western blot or an enzyme-linked immunosorbent assay (ELISA). Prior disruption of IGFBP-4 complexes using an inhibitor of IGFBP-4 binding IGF-II may be required for some of these assays. An example of one such inhibitor is IGFBP-3. Associated kits, uses, and antibodies are also described.

### 1. Definitions

“Intrauterine growth restriction” (IUGR), also known as “fetal growth restriction” (FGR), is a leading cause of perinatal mortality and morbidity in the developed world. Failure of a fetus to achieve its growth potential is associated with increased risks of perinatal complications and is linked to a higher incidence of adult-onset diseases such as hypertension, diabetes, hyperlipidemia and cardiovascular diseases (1). It is important to note that IUGR can occur in the presence or absence of pre-eclampsia (49). It is usually diagnosed by ultrasound (umbilical artery and ductus veniosus Doppler), and the only clinical management strategy for IUGR is delivery.

“Sample”, as used herein refers to a biological sample from a person which can be analyzed or tested, including tissue samples (such as biopsies), and fluid samples. Fluid samples may include blood, blood-derived products, plasma, saliva, serum, urine, sweat, mucous, amniotic fluid, etc.

“Biomarker”, as used herein, reference to any biological molecule that is associated with a disease state, condition, trait, or risk thereof. Biomarkers may encompass protein, DNA, RNA, polysaccharides, metabolites, or other biological molecules. A “biomarker”, as used herein, may also be a genetic variant in a biological molecule, such as a polymorphism or mutation detected in DNA, RNA, or protein using widely known methods.

“Genetic variant”, as used herein encompasses a sequence change at a position in a gene, a regulatory element thereof, or in non-coding sequence which may be silent or result in a corresponding amino acid change. A “genetic variant” may be defined as sequence difference compared to the most abundance sequence within population of control individuals (and taking into account ethnic breakdown of that population, which may impact the frequency of genetic variants). A “genetic variant” of may also simply reference a specific sequence at a highly polymorphic site. Genetic variants may be associated with a disease, a condition, or a trait; or a risk of developing one of these. A genetic variant may increase, decrease, or have no effect on the expression or activity of a protein. Genetic variants may be detected using standard techniques known in the art, including (but not limited to) sequencing, allele-specific hybridization, allele-specific PCR, restriction fragment analysis primer extension, molecular beacons, flap endonuclease-based methodologies, etc.

Generally, gene and protein names (IGFBP-4, IGF-II, IGFBP-3, IGFBP-4, IGF-II, IGFBP-3, etc.) referenced herein refer to proteins when they appears non-italicized, and to nucleic acids when italicized. Proteins also encompass detectable fragments and isoforms of a recited protein. Nucleic acids also encompass detectable fragments, isoforms, and splice variants thereof. Proteins and gene/mRNA sequences also encompass variants thereof, such as variants captured by various sequences deposited in GenBank (http://www.ncbi.nlm.nih.gov/gquery/), including the data on single nucleotide polymorphisms (SNPs) held in dbSNP (http://www.ncbi.nlm.nih.gov/snp/) and elsewhere.

“IGFBP-4” is a protein encoded by the IGFBP-4 gene, and is a member of the insulin-like growth factor binding protein (IGFBP) family and encodes a protein with an IGFBP domain and a thyroglobulin type-I domain. The protein binds both insulin-like growth factors (IGFs) I and II and circulates in the plasma in both glycosylated and non-glycosylated forms. Binding of this protein prolongs the half-life of the IGFs and alters their interaction with cell surface receptors. Further information on the gene may be found under Online Medelian Inheritance in Man (OMIM; http://www.ncbi.nlm.nih.gov/omim/) entry *146733 and the gene's mRNA sequence is set out in GenBank Accession NM_{—}001552, both which are expressly incorporated herein by reference in their entirety.

“IGFBP-3” likewise is encoded by the IGFBP-3 gene, and is a member of the IGFBP family. The protein comprises an IGFBP domain and a thyroglobulin type-I domain. The protein forms a ternary complex with insulin-like growth factor acid-labile subunit (IGFALS) and either insulin-like growth factor (IGF) I or II. In this form, it circulates in the plasma, prolonging the half-life of IGFs and altering their interaction with cell surface receptors. Alternate transcriptional splice variants, encoding different isoforms, have been characterized. Further information on the gene may be found under OMIM entry *146732 and the gene's genomic sequence is set out in GenBank Accession NG_{—}011508, both which are expressly incorporated herein by reference in their entirety.

“IGF-II” is one of three protein hormones that share structural similarity to insulin. The major role of IGF2 is as a growth promoting hormone during gestation. IGF2 may also bind to the IGF-2 receptor (also called the cation-independent mannose 6-phosphate receptor), which acts as a signaling antagonist; that is, to prevent IGF2 responses. It exerts its effects by binding to the IGF-1 receptor. IGF2 may also bind to the IGF-2 receptor (also called the cation-independent mannose 6-phosphate receptor), which acts as a signaling antagonist; that is, to prevent IGF2 responses. In the process of Folliculogenesis, IGF2 is created by Theca cells to act in an autocrine manner on the theca cells themselves, and in a paracrine manner on Granulosa cells in the ovary. IGF2 promotes granulosa cell proliferation during the follicular phase of the menstrual cycle, acting alongside Follicle Stimulating Hormone (FSH). After ovulation has occurred, IGF-2 promotes progesterone secretion during the luteal phase of the menstrual cycle together with Luteinizing Hormone (LH). Thus, IGF2 acts as a Co-hormone together with both FSH and LH. Further information on the gene may be found under OMIM entry *147470 and the gene's genomic sequence is set out in Gen Bank Accession NG_{—}008849, both which are expressly incorporated herein by reference in their entirety.

“IGFBP-4 level”, as used herein, indicates the amount of IGFBP4 protein.

“Measurement indicative of IGFBP-4 level” is any measurement which may directly or indirectly provide an indicator or proxy of IGFBP-4 protein level. Such measurements may include, for example, direct measurement of the protein (or a fragment or isoform thereof) in a sample, or measurement of an mRNA (or a fragment or isoform thereof) which is generally correlated with protein level. Such measurements may also include measurements of genetic variants, such as a single nucleotide polymorphism (SNP) which are correlated with increased mRNA and/or protein levels.

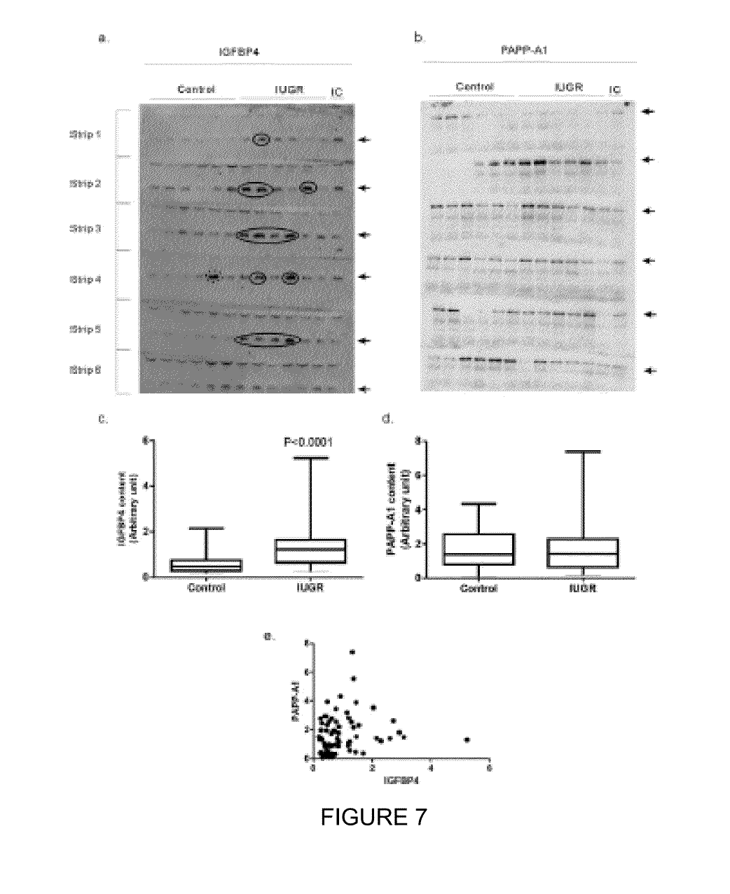

“Elevated level” or “increased level”, as used herein with respect to the level of a biomarker, such as IGFBP-4, indicates that the measured level is high within the spectrum of levels measured amongst the general population; or compared to measurements in those deemed not to posses an elevated risk of a particular disease or conditions. “High” may indicate a measured level at or above the 50^{th}, 55^{th}, 80^{th}, 85^{th}, 70^{th}, 75^{th}, 80^{th}, 85^{th}, 90^{th} or 95^{th} percentile. A skilled person would be able to analyze biological data, such as that which appears in FIG. 7, and set a threshold value is to produce a test optimized for desirable sensitivity and specificity values, given information regarding the intended purpose of the test. In some instances, it may be desirable to set a threshold to determine what constitutes an “elevated level” in order to yield a more specific test, i.e. one in which those who possess an “elevated level” are very highly enriched for individuals who have or are at risk of developing a particular condition, at the cost of missing some such individual who are below the threshold value. In other contexts, it may be more desirable to have a more sensitive test, in which the group of those deemed to possess an “elevated level” captures a greater number of those who are at risk of developing a particular condition, albeit at a reduced specificity.

“Increased expression” means that there are increased steady-state levels of a given biomarker, such as an mRNA or a protein. The increase may be due to increased transcription/translation, increased stability, or reduced degradation, for example.

“Increased activity” means that at least one biological activity of a biological molecule is higher than normal. For example, in some instances increased expression of may result in increased activity. In other instances, a sequence change may result in a protein having high activity.

“Elevated risk” or “increased risk”, as used herein, indicates that within particular test group deemed to possess an elevated probability of having (or developing in future) a particular disease or condition, i.e. within a group deemed to possess an “elevated risk”, there will be a statitistically significant greater proportion of cases of said disease or condition (or a greater proportion of individuals will go on to develop said disease or condition) (i) relative to the general population; or (ii) relative to a population deemed not to possess an “elevated risk”.

“Protein” and “mRNA”, as defined herein (and particularly in contexts pertaining to their detection) include fragments of corresponding full proteins and mRNAs which may be detected and/or measured using standard techniques, such as (for proteins) immunoassays, including ELISAs, Western blotting, etc.; and (for mRNAs) RT-PCT, primer extension, and hybridization-based assays, including Northern blot analysis, array-based hybridization methods, etc.

“Antibody” as defined herein is intended to include monoclonal antibodies, polyclonal antibodies, chimeric antibodies, humanized antibodies, human antibodies, mouse antibodies, etc. and fragments thereof which specifically bind to a protein, such as IGFBP-4. Antibodies can be fragmented using conventional techniques and the fragments screened for utility. For example, fragments can be generated by treating an antibody with pepsin. The resulting fragment can be further treated to reduce disulfide bridges. “Chimeric antibodies” are antibodies comprises sequences of two different origins, such as those resulting from the combination of a variable non-human animal peptide region and a constant human peptide region are also contemplated within the scope of the invention. Chimeric antibody molecules can include, for example, the antigen binding domain from an antibody of a mouse, rat, or other species with a constant human peptide region. Conventional methods may be used to make chimeric antibodies containing the immunoglobulin variable region which recognizes IGFBP-4 (see, for example, 58-63). “Humanized antibodies” can be generated by humanizing other antibodies, for instance by producing human constant region chimeras, in which parts of the variable regions—particularly the conserved framework regions of the antigen-binding domain—are of human origin and only the hypervariable regions are of non-human origin. Such molecules may be made by techniques known in the art (64-68). Humanized antibodies can also be commercially produced (Scotgen Limited, 2 Holly Road, Twickenham, Middlesex, Great. Britain). Antibodies may include those which are commercially available, including.

“Inhibitor”, as used herein in the context of inhibiting protein complex formation, is a molecule which prevents protein complexes from forming and/or promotes their dissociation. An inhibitor may be non-competitive or competitive. “Competitive inhibition” means that inhibitor competes with another molecule for binding e.g. (i) by binding to a particular target site on a target and preventing binding of the other molecule (e.g. by steric hindrance), (ii) by binding to another site and blocking the target site such that the other molecule cannot bind (e.g. by steric hindrance), or (iii) by binding to another site and inducing a conformational change at the target site such that the other molecule cannot bind.

“About”, as used herein, indicates a range of plus or minus 5% based on a stated numerical value.

Experimental techniques known to one of skill in the art include (but are not limited to) those taught in Sambrook, et al. in Molecular Cloning: A Laboratory Manual (Third Edition) (Cold Spring Harbor Laboratory Press).

### 2. Method of Determining Risk of IUGR/FGR

In one aspect there is provided a method of determining risk of intrauterine growth restriction (IUGR) during pregnancy comprising the steps of: obtaining a sample from a pregnant woman; taking a measurement indicative of an IGFBP-4 level in the sample; and determining that an increased risk of IUGR exists if the IGFBP-4 level is elevated.

The sample may comprise blood, serum, plasma, urine, or amniotic fluid. In one exemplary embodiment, the sample comprises serum.

Generally, the sample is taken during pregnancy. The sample may be obtained during the first, second, or third trimester. In one embodiment, the sample is obtained during the first or second trimester of pregnancy. In one embodiment, it is obtained during the first trimester. For instance, the sample may comprise blood, serum, plasma, urine or amniotic fluid taken during the first trimester of pregnancy. In one exemplary embodiment, the sample comprises serum obtained during the first trimester of pregnancy. In another exemplary embodiment, the sample is one obtained for the current routine screening, e.g. for Down syndrome.

The method may be particularly useful for determining risk of IUGR in women having a personal history or family history of one or more of the following: IUGR; pre-eclampsia; eclampsia; HELLP syndrome; hypertension; vascular disease; auto-immune disease; renal disease; or diabetes. In one embodiment, the pregnant woman may have a personal or family history of IUGR. In one specific embodiment, the pregnant woman may have a personal history of IUGR.

The measurement indicative of IGFBP-4 level may be a measurement of IGFBP-4 protein, IGFBP-4 mRNA, or measurement or detection of a genetic variant thereof which leads to increased expression or activity.

In one embodiment, IGFBP-4 mRNA may be measured. A skilled person would be well aware of techniques for measuring mRNA levels, such as Northern blot analysis, RT-PCR, real time RT-PCR, array-based hybridization methods, etc.

In one embodiment, the measurement is a measurement of IGFGP-4 protein. The protein may be detected through use of an antibody. The antibody may be a commercially available antibody, such as sc-6005 (Santa Cruz Biotechnology, Santa Cruz, Calif.) or ER-14-0734 (RayBiotech, Inc 3607 parkway Lane, Suit 200, Norcross Ga.). The protein may be detected by immunoblotting methods, such as Western blot analysis, including multi-strip Western blot analysis. The protein may also be detected through use of an enzyme-linked immunosorbent assay (ELISA).

In some embodiments, it may be necessary to disrupt IGFBP-4 complexation with other proteins in order to facilitate detection. An inhibitor may be used to disrupts complexes comprising IGFBP-4. For instance, IGFBP-4 may complex with IGF-II, thereby inhibiting detection of IGFBP-4 with some antibodies. In such circumstances, it is possible to disrupt these complexes to facilitate detection through use of an inhibitor which prevents IGFBP-4 from binding to IGF-II or which otherwise promotes dissociation of this complex. Thus, in some embodiments, the method comprises disrupting complexation with an inhibitor prior to taking the measurement. In some embodiments, the inhibitor is a competitive inhibitor which competes with IGFBP-4 for binding to IGF-II. In one specific example, the competitive inhibitor may be IGFBP-3, which binds to IGF-II with higher affinitity than IGFBP-4.

As noted, the IGFBP-4 level may be considered “elevated” if it is above a threshold which is the 50th, 60th, 70th, 80th, 90th, or 95th percentile. A skilled person would recognize that setting different cutoff thresholds to define what constitutes an “elevated” level of IGFBP-4 would result in a test having different sensitivity and specificity parameters. In some clinical contexts, it may be beneficial to increase sensitivity at the expense of specificity. In other contexts, increased specificity may be more desirable. On the basis of the teaching herein (e.g. the data in FIG. 7), a skilled person would be able to adjust the these parameters to tailor a method to suit specific needs.

The level may be considered “elevated” if it is above the 50^{th}, 55^{th}, 60^{th}, 65^{th}, 70^{th}, 75^{th}, 80^{th}, 85^{th}, 90^{th}, or 95^{th} percentile. It may be considered “elevated” if it is above about the 50^{th} percentile. It may be considered “elevated” if it is above about the 60^{th} percentile. It may be considered “elevated” if it is above about the 70^{th} percentile. In one exemplary embodiment, a level is considered to be considered “elevated” if it is above about the 80th percentile.

The associated sensitivity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the sensitivity is about 38%.

The associated specificity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the specificity is about 97%.

The positive predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the positive predictive value is about 93%.

The negative predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the negative predictive value is about 74%.

### 3. Kit for Determining Risk of IUGR/FGR

In another aspect, there is provided a kit for determining a risk of intrauterine growth restriction (IUGR) during pregnancy from a sample obtained from a pregnant woman comprising: reagents for taking a measurement indicative of an IGFBP-4 level in said sample; and instructions for use, wherein an increased risk of IUGR is determined if the level of IGFBP-4 is elevated.

The sample may comprise blood, serum, plasma, urine, or amniotic fluid. In one exemplary embodiment, the sample comprises serum.

Generally, the kit is for determining risk of IUGR during pregnancy. The kit may be for determining risk of IUGR during the first, second, or third trimester. In one embodiment, the kit if for determining risk of IUGR during the first or second trimester of pregnancy. In one embodiment, the kit is for determining risk of IUGR during the first trimester. For instance, the sample may comprise blood, serum, plasma, urine or amniotic fluid obtained during the first trimester of pregnancy. In one exemplary embodiment, the sample comprises serum obtained during the first trimester of pregnancy. In another exemplary embodiment, the sample is one obtained for the current routine screening, e.g. for Down syndrome.

The kit may be particularly useful for determining risk of IUGR in women having a personal history or family history of one or more of the following: IUGR; pre-eclampsia; eclampsia; HELLP syndrome; hypertension; vascular disease; auto-immune disease; renal disease; or diabetes. In one embodiment, the kit is for determining risk of IUGR in pregnant woman having a personal or family history of IUGR. In one specific embodiment, the kit is for determining risk of IUGR in a pregnant woman having a personal history of IUGR.

The reagents may be for measuring IGFBP-4 protein, IGFBP-4 mRNA, or detecting/measuring a genetic variant of IGFBP-4 which leads to increased expression or activity.

In one embodiment, the reagents are for measuring IGFBP-4 mRNA levels. A skilled person would be well aware of techniques for measuring mRNA levels, such as Northern blot analysis, RT-PCR, real time RT-PCR, array-based hybridization methods, etc.; and the necessary reagents used in these methods.

In one embodiment, the reagents are for measuring of IGFGP-4 protein. The reagents may comprise and antibody for detecting IGFBP-4. The antibody may be a commercially available antibody, such as sc-6005 (Santa Cruz Biotechnology, Santa Cruz, Calif.) or ER-14-0734 (RayBiotech, Inc 3607 parkway Lane, Suit 200, Norcross Ga.). The reagents may be for detecting the protein via immunoblotting methods, such as Western blot analysis, including multi-strip Western blot analysis. The reagents may also be for detecting the protein through an ELISA.

In some embodiments, it may be necessary to disrupt IGFBP-4 complexation with other proteins in order to facilitate detection. For example, an inhibitor may be used to disrupt complexes comprising IGFBP-4. For instance, IGFBP-4 may complex with IGF-II, thereby inhibiting detection with some antibodies. In such circumstances, it is possible to disrupt these complexes to facilitate detection through use of reagents comprising an inhibitor which prevents IGFBP-4 from binding to IGF-II or which otherwise promotes dissociation of this complex. Thus, in some embodiments, the reagents comprise an inhibitor for disrupting complexation prior to taking the measurement. In some embodiments, the inhibitor is a competitive inhibitor which competes with IGFBP-4 for binding to IGF-II. In one specific example, the competitive inhibitor may be IGFBP-3, which binds to IGF-II with higher affinity than IGFBP-4.

As noted, the IGFBP-4 level may be considered “elevated” if it is above a threshold which is the 50th, 60th, 70th, 80th, 90th, or 95th percentile. A skilled person would recognize that setting different cutoff thresholds to define what constitutes an “elevated” level of IGFBP-4 would result in a test having different sensitivity and specificity parameters. In some clinical contexts, it may be beneficial to increase sensitivity at the expense of specificity. In other contexts, increased specificity may be more desirable. The kit instructions may recite parameters to allow a practitioner to tailor the test to suit specific needs.

The level may be considered “elevated” if it is above the 50^{th}, 55^{th}, 60^{th}, 65^{th}, 70^{th}, 75^{th}, 80^{th}, 85^{th}, 90^{th}, or 95^{th} percentile. It may be considered “elevated” if it is above about the 50^{th} percentile. It may be considered “elevated” if it is above about the 60^{th} percentile. It may be considered “elevated” if it is above about the 70^{th} percentile. In one exemplary embodiment, a level is considered to be considered “elevated” if it is above about the 80th percentile.

The associated sensitivity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the sensitivity is about 38%.

The associated specificity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the specificity is about 97%.

The positive predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the positive predictive value is about 93%.

The negative predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the negative predictive value is about 74%.

### 4. Use of a Measurement Indicative of IGFBP-4 Levels for Determining Risk of IUGR/FGR

In another aspect, there is provided a use of a measurement indicative of an IGFBP-4 level in a sample obtained from a pregnant woman for determining a risk of intrauterine growth restriction (IUGR) during pregnancy, wherein an increased IGFBP-4 level is indicative of an increased risk of IUGR.

The sample may comprise blood, serum, plasma, urine, or amniotic fluid. In one exemplary embodiment, the sample comprises serum.

Generally, the sample is obtained during pregnancy. The sample may be obtained during the first, second, or third trimester. In one embodiment, the sample is obtained during the first or second trimester of pregnancy. In one embodiment, it may be obtained during the first trimester. For instance, the sample may comprise blood, serum, plasma, urine or amniotic fluid taken during the first trimester of pregnancy. In one exemplary embodiment, the sample comprises serum obtained during the first trimester of pregnancy. In another exemplary embodiment, the sample is one obtained for the current routine screening, e.g. for Down syndrome.

The method may be particularly useful for determining risk of IUGR in women having a personal history or family history of one or more of the following: IUGR; pre-eclampsia; eclampsia; HELLP syndrome; hypertension; vascular disease; auto-immune disease; renal disease; or diabetes. In one embodiment, the pregnant woman may have a personal or family history of IUGR. In one specific embodiment, the pregnant woman may have a personal history of IUGR.

The measurement indicative of IGFBP-4 level may be a measurement of IGFBP-4 protein, IGFBP-4 mRNA, or a measurement or detection of a genetic variant of IGFBP-4 which leads to increased expression or activity.

In one embodiment, IGFBP-4 mRNA may be measured. A skilled person would be well aware of techniques for measuring mRNA levels, such as Northern blot analysis, RT-PCR, real time RT-PCR, array-based hybridization methods, etc.

In one embodiment, the measurement is a measurement of IGFGP-4 protein. The protein may be detected through use of an antibody. The antibody may be a commercially available antibody, such as sc-6005 (Santa Cruz Biotechnology, Santa Cruz, Calif.) or ER-14-0734 (RayBiotech, Inc 3607 parkway Lane, Suit 200, Norcross Ga.). The protein may be detected by immunoblotting methods, such as Western blot analysis, including multi-strip Western blot analysis. The protein may be detected through use of an ELISA.

In some embodiments, it may be necessary to disrupt IGFBP-4 complexation with other proteins in order to facilitate detection. For example, an inhibitor could be used to disrupt complexes comprising IGFBP-4. For instance, IGFBP-4 may complex with IGF-II, thereby inhibiting detection with some antibodies. In such circumstances, it is possible to disrupt these complexes to facilitate detection by using of an inhibitor which prevents IGFBP-4 from binding to IGF-II or which otherwise promotes dissociation of this complex. Thus, in some embodiments, the use comprises disrupting complexation with an inhibitor prior to taking the measurement. In some embodiments, the inhibitor is a competitive inhibitor which competes with IGFBP-4 for binding to IGF-II. In one specific example, the competitive inhibitor may be IGFBP-3, which binds to IGF-II with higher affinity than IGFBP-4.

As noted, the IGFBP-4 level may be considered “elevated” if it is above a threshold which is the 50th, 60th, 70th, 80th, 90th, or 95th percentile. A skilled person would recognize that setting different cutoff thresholds to define what constitutes an “elevated” level of IGFBP-4 would result in a test having different sensitivity and specificity parameters. In some clinical contexts, it may be beneficial to increase sensitivity at the expense of specificity. In other contexts, increased specificity may be more desirable. On the basis of the teaching herein, a skilled person would be able to adjust the these parameters to tailor a method to suit specific needs.

The level may be considered “elevated” if it is above the 50^{th}, 55^{th}, 60^{th}, 65^{th}, 70^{th}, 75^{th}, 80^{th}, 85^{th}, 90^{th}, or 95^{th} percentile. It may be considered “elevated” if it is above about the 50^{th} percentile. It may be considered “elevated” if it is above about the 60^{th} percentile. It may be considered “elevated” if it is above about the 70^{th} percentile. In one exemplary embodiment, a level is considered to be considered “elevated” if it is above about the 80th percentile.

The associated sensitivity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the sensitivity is about 38%.

The associated specificity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the specificity is about 97%.

The positive predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the positive predictive value is about 93%.

The negative predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the negative predictive value is about 74%.

### 5. Antibody for Determining IGFBP-4 Level in a Sample

In one aspect, there is provided a use of an antibody specific to IGFBP-4 for determining an IGFBP-4 level in a sample obtained from a pregnant woman, and determining risk of intrauterine growth restriction (IUGR), wherein an increased level of IGFBP-4 is indicative of increased risk of IUGR.

In another aspect, there is provided an antibody for use in determining an IGFBP-4 level in a sample obtained from a pregnant woman and determining a risk of intrauterine growth restriction (IUGR), wherein an increased level of IGFBP-4 is indicative of an increased risk of IUGR.

The sample may comprise blood, serum, plasma, urine, or amniotic fluid. In one exemplary embodiment, the sample comprises serum.

Generally, the sample is one taken during pregnancy. The sample may be obtained during the first, second, or third trimester. The sample may also be obtained during the first or second trimester. In one embodiment, the sample is obtained during the first trimester of pregnancy. For instance, the sample may comprise blood, serum, plasma, urine or amniotic fluid obtained during the first trimester of pregnancy. In one exemplary embodiment, the sample comprises serum obtained during the first trimester of pregnancy. In another exemplary embodiment, the sample is one obtained for the current routine screening, e.g. for Down syndrome.

The use of the antibody may be for determining risk of IUGR in women having a personal history or family history of one or more of the following: IUGR; pre-eclampsia; eclampsia; HELLP syndrome; hypertension; vascular disease; auto-immune disease; renal disease; or diabetes. In one embodiment, the pregnant woman may have a personal or family history of IUGR. In one specific embodiment, the pregnant woman may have a personal history of IUGR.

The antibody may be a commercially available antibody, such as sc-6005 (Santa Cruz Biotechnology, Santa Cruz, Calif.) or ER-14-0734 (RayBiotech, Inc 3607 parkway Lane, Suit 200, Norcross Ga.). The protein may be detected by immunoblotting methods, such as Western blot analysis, including multi-strip Western blot analysis. The protein may be detected through an ELISA.

In some embodiments, it may be necessary to disrupt IGFBP-4 complexation with other proteins in order to facilitate detection, prior to using the antibody. For example, an inhibitor could be used to disrupt complexes comprising IGFBP-4. For instance, IGFBP-4 may complex with IGF-II, thereby inhibiting detection with some antibodies. In such circumstances, it is possible to disrupt these complexes to facilitate detection through use of an inhibitor which prevents IGFBP-4 from binding to IGF-II or which otherwise promotes dissociation of this complex. Thus, in some embodiments, the method comprises disrupting complexation with an inhibitor prior to taking the measurement. In some embodiments, the inhibitor is a competitive inhibitor which competes with IGFBP-4 for binding to IGF-II. In one specific example, the competitive inhibitor may be IGFBP-3, which binds to IGF-II with higher affinitity than IGFBP-4.

As noted, the IGFBP-4 level may be considered “elevated” if it is above a threshold which is the 50th, 60th, 70th, 80th, 90th, or 95th percentile. A skilled person would recognize that setting different cutoff thresholds to define what constitutes an “elevated” level of IGFBP-4 would result in a test having different sensitivity and specificity parameters. In some clinical contexts, it may be beneficial to increase sensitivity at the expense of specificity. In other contexts, increased specificity may be more desirable. On the basis of the teaching herein, a skilled person would be able to adjust the these parameters to tailor a method to suit specific needs.

The level may be considered “elevated” if it is above the 50^{th}, 55^{th}, 60^{th}, 65^{th}, 70^{th}, 75^{th}, 80^{th}, 85^{th}, 90^{th}, or 95^{th} percentile. It may be considered “elevated” if it is above about the 50^{th} percentile. It may be considered “elevated” if it is above about the 60^{th} percentile. It may be considered “elevated” if it is above about the 70^{th} percentile. In one exemplary embodiment, a level is considered to be considered “elevated” if it is above about the 80th percentile.

The associated sensitivity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the sensitivity is about 38%.

The associated specificity may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the specificity is about 97%.

The positive predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the positive predictive value is about 93%.

The negative predictive value may be about 25%, about 30%, about 35%, about 40%, about 45%, about 50%, about 55%, about 60%, about 65%, about 70%, about 75%, about 80%, about 85%, about 90%, or about 95%. In one exemplary embodiment, the negative predictive value is about 74%.

### 7. Other Embodiments

There is provided a method of determining risk of intrauterine growth restriction (IUGR) during pregnancy comprising the steps of: obtaining a sample from a pregnant woman; taking a measurement indicative of an IGFBP-4 level in the sample; and determining that an increased risk of IUGR exists if the IGFBP-4 level is elevated.

The sample may comprise blood, serum, plasma, urine, or amniotic fluid.

The sample may comprise serum.

The sample may be obtained during the first trimester of pregnancy.

The pregnant woman may be a woman having a personal or family history of IUGR; pre-eclampsia; eclampsia; HELLP syndrome; hypertension; vascular disease; auto-immune disease; renal disease; or diabetes.

The measurement may be a measurement of IGFBP-4 protein, IGFBP-4 mRNA, or a genetic variant of IGFBP-4 which leads to increased expression or activity.

The measurement may be a measurement of IGFBP-4 protein.

The measurement may be made by an enzyme-linked immunosorbent assay (ELISA).

Prior to the step of taking a measurement, an inhibitor may be added to disrupt complexes comprising IGFBP-4. The complexes may be complexes comprising IGFBP-4 and IGF-II. The inhibitor may be a competitive inhibitor. The competitive inhibitor may comprises IGFBP-3.

The IGFBP-4 level may be considered to be elevated if it is higher than eightieth percentile. The sensitivity of the method may be about 38% or the specificity may be about 97%. The positive predictive value of the method may be about 93% or the negative predictive value may be about 74%.

There is provided a kit for determining a risk of intrauterine growth restriction (IUGR) during pregnancy from a sample obtained from a pregnant woman comprising: reagents for taking a measurement indicative of an IGFBP-4 level in said sample; and instructions for use, wherein an increased risk of IUGR is determined if the level of IGFBP-4 is elevated.

The kit may be for determining the risk of intrauterine growth restriction during the first trimester of pregnancy.

The pregnant woman may be a woman having a personal or family history of IUGR; pre-eclampsia; eclampsia; HELLP syndrome; hypertension; vascular disease; auto-immune disease; renal disease; or diabetes.

The reagents may be for measuring IGFBP-4 protein, IGFBP-4 mRNA, or a genetic variant of IGFBP-4 which leads to increased expression or activity.

The reagents may be for measuring IGFBP-4 protein.

The reagents may be for measuring IGFBP-4 protein by an enzyme-linked immunosorbent assay (ELISA).

The reagents may comprise an inhibitor for disrupting complexes comprising IGFBP-4. The complexes may be complexes comprising IGFBP-4 and IGF-II. The inhibitor may comprise a competitive inhibitor. The competitive inhibitor may comprise IGFBP-3.

The instructions may indicate that the increased risk is present if the level of IGFBP-4 is higher than the eightieth percentile.

There is provided a use of a measurement indicative of an IGFBP-4 level in a sample obtained from a pregnant woman for determining a risk of intrauterine growth restriction (IUGR) during pregnancy, wherein an increased IGFBP-4 level is indicative of an increased risk of IUGR.

The sample may comprise blood, serum, plasma, urine, or amniotic fluid.

The sample may comprise serum.

The sample may be from during the first trimester of pregnancy.

The pregnant woman may be a woman having a personal or family history of IUGR; pre-eclampsia; eclampsia; HELLP syndrome; hypertension; vascular disease; auto-immune disease; renal disease; or diabetes.

The measurement may be a measurement of IGFBP-4 protein, IGFBP-4 mRNA, or a genetic variant of IGFBP-4 which leads to increased expression or activity.

The measurement may be a measurement of IGFBP-4 protein.

The measurement may be made by an enzyme-linked immunosorbent assay (ELISA).

The measurement may be carried out using a sample to which an inhibitor has been added to disrupt complexes comprising IGFBP-4. The complexes may be complexes comprising IGFBP-4 and IGF-II. The inhibitor may be a competitive inhibitor. The competitive inhibitor may comprises IGFBP-3.

The IGFBP-4 level may be considered to be elevated if it is higher than eightieth percentile.

There is provided a use of an antibody specific to IGFBP-4 for determining an IGFBP-4 level in a sample obtained from a pregnant woman, and determining risk of intrauterine growth restriction (IUGR), wherein an increased level of IGFBP-4 is indicative of increased risk of IUGR.

The sample may comprise blood, serum, plasma, urine, or amniotic fluid.

The sample may comprise serum.

The sample may be from during the first trimester of pregnancy.

The pregnant woman may be a woman having a personal or family history of IUGR; pre-eclampsia; eclampsia; HELLP syndrome; hypertension; vascular disease; auto-immune disease; renal disease; or diabetes.

The antibody may be used in an enzyme-linked immunosorbent assay (ELISA).

The antibody may be used with a sample into which an inhibitor of complexes comprising IGFBP-4. The complexes may be complexes comprising IGFBP-4 and IGF-II. The inhibitor may comprises a competitive inhibitor. The competitive inhibitor may be IGFBP-3.

The increased level of IGFBP-4 may be a level that is higher than the eightieth percentile.

There is provided an antibody for use in determining an IGFBP-4 level in a sample obtained from a pregnant woman and determining a risk of intrauterine growth restriction (IUGR), wherein an increased level of IGFBP-4 is indicative of an increased risk of IUGR.

The sample may comprise blood, serum, plasma, urine, or amniotic fluid.

The sample may comprise serum.

The sample may be from during the first trimester of pregnancy.

The pregnant woman may be a woman having a personal or family history of IUGR; pre-eclampsia; eclampsia; HELLP syndrome; hypertension; vascular disease; auto-immune disease; renal disease; or diabetes.

The antibody may be used in an enzyme-linked immunosorbent assay (ELISA).

The antibody may be used with a sample into which an inhibitor of complexes comprising IGFBP-4. The complexes may be complexes comprising IGFBP-4 and IGF-II. The inhibitor may comprises a competitive inhibitor. The competitive inhibitor may be IGFBP-3.

The increased level of IGFBP-4 may be a level that is higher than the eightieth percentile.

In one aspect, there is provided a use of a measurement indicative of an IGFBP-4 level in a sample obtained from a pregnant woman for determining a risk of intrauterine growth restriction (IUGR) during pregnancy, wherein an increased IGFBP-4 level is indicative of an increased risk of IUGR.

In one aspect, there is provided a use of an antibody specific to IGFBP-4 for determining an IGFBP-4 level in a sample obtained from a pregnant woman, and determining risk of intrauterine growth restriction (IUGR), wherein an increased level of IGFBP-4 is indicative of increased risk of IUGR.

In one aspect, there is provided an antibody for use in determining an IGFBP-4 level in a sample obtained from a pregnant woman and determining a risk of intrauterine growth restriction (IUGR), wherein an increased level of IGFBP-4 is indicative of an increased risk of IUGR.

### EXAMPLES

### Example 1

### Identification of IGFBP-4 as a Maternal Serum Biomarker for IUGR During Early Gestation

### A. Material and Methods

### Specimens for Immunohistochemistry

After obtaining informed consent and approval from the local Research Ethics Board, placental tissues from the maternal-fetal junctional zones were collected from three healthy women during early pregnancy (10-13 weeks gestation) following surgical terminations. Dating and viability were confirmed by a first trimester ultrasound. Upon collection, samples were fixed overnight in 4% (v/v) paraformaldehyde-PBS (phosphate buffered saline), dehydrated through graded series of ethanol and embedded in paraffin. Paraffin sections (4-5 μm) were cut and mounted on pre-cleaned and charged slides.

### Immunohistochemistry

Serial sections were deparaffinized in xylene and rehydrated with a graded series of ethanol. Single immunostains were performed with the DAKO LSAB Kit (DAKO Corporation, Mississauga, Ontario, Canada). Briefly, the sections were heated by microwave (15 min) in 0.01M citrate buffer (pH 6.0) to retrieve antigens and incubated in PBS with 3% H2O2 (10 min.) to inactivate endogenous peroxidase activity, and subsequently with blocking solution (10 min.) (DAKO Corporation, Mississauga, Ontario, Canada). Four adjacent sections of maternal-fetal interface tissues were then incubated with either anti-IGFBP-4 (sc-6005, Santa Cruz Biotechnology, Santa Cruz, Calif.), anti-PAPP-A (#AF2487, R&D system, Minneapolis, Minn.), anti-vimentin (#sc-7557, Santa Cruz Biotechnology, Santa Cruz, Calif. as decidual stroma cell marker) and anti-cytokeratin 18 antibody (#sc-6259, Santa Cruz Biotechnology, Santa Cruz, Calif., as a extravillous trophoblast marker) respectively. The sections were exposed to mouse IgG and goat IgG, respectively, instead of primary antibody, thus serving as negative controls. After washing, biotinylated secondary antibody and streptavidin conjugated with HRP were applied to sections. Specific immunoreactvities were displayed with 3-amino-9-ethylcarbazole (AEC) substrate and nuclear counter-stained with hematoxylin. All immuno-signals were recorded by ScanScope™ [Aperio, Vista, Calif.]. Sections stained for PAPP-A were additionally dual stained for cytokeratin 18, to detect syncytiotrophoblast. These sections were therefore de-stained with 70% ethanol/1% HCl for several minutes to remove all visible red color pigment. To avoid interference from previous agents with HRP activity, the sections were again boiled in citrate buffer for 10 minutes prior to staining with anti-cytokeratin 18 antibody before incubation with anti-cytokeratin 18 antibody (#sc-6259, Santa Cruz Biotechnology, Santa Cruz, Calif.).

Determination of IGF-II, IGFBPs and IGF-II complex in HTR8/SVneo conditioned media.

HTR8/SVneo cells (a generous gift of Dr. C. Graham, Kingston, Ontario, Canada), which are representative of human extravillous trophoblast lineage, were cultured in serum-free medium for 48 hours. The conditioned media was collected and concentrated (20 times) with Amicon Ultra centrifugal filter devices (ultracel-3k, Milipore Corporation, Billerica, Mass.). An aliquot of 20 μl concentrated media was applied to Western ligand blot (WLB) and Western blot (WB) analysis under non-reducing condition as described previously (18). IGFBPs were determined by WLB and the identification of specific IGFBPs was confirmed by WB with IGFBP2 (#06-107, Upstate), IGFBP3 (GroPep Ltd., Adelaide, Australia) and IGFBP-4 (sc-6005) (Santa Cruz Biotechnology, Santa Cruz, Calif.) antibodies respectively. IGF-II was also examined by WB with anti-IGF-II antibody (cloneS1F2, Upstate, Lake Placid, N.Y.). To assess whether IGFBP-4 could form a complex with IGF-II, aliquots of 20 μl conditioned media with 2 mM Disuccinimidyl suberate (DSS, Thermo Fisher Scientific, Rockford, Ill.) were incubated at 4° C. overnight with shaking to promote cross-linking. Relative quantities of IGF-II and IGF-II-IGFBP-4 complexes were determined by WB using anti-IGF-II antibody, and IGFBP-4 content with or without cross-link were also determined by WB.

Serum Samples from Healthy Non-Pregnant and Pregnant Women During Early, Mid and Late Gestation

Serum samples were obtained from healthy non-pregnant women, and pregnant women during the 1st trimester (7-10 weeks, N=6), 2nd trimester (17-21 weeks, N=6) and 3rd trimester (37-40 weeks, N=6)) at the Third Hospital of Hebei Medical University, Shijiazhuang, Hebei, China, as described above. This study was approved by the local ethics committees and written informed consent was obtained from all donors. Samples were stored at −80° C. for later batch analysis.

### Sample Collection in Nested Case-Control

As approved by the local institutional Research Ethics Board, study patients were selected from a large prospective cohort of over 8000 women between 12-13.6 weeks gestation (OaK birth cohort (50)). For each of these patients informed consent was obtained, and peripheral blood was collected by venipuncture. The blood was allowed to coagulate at 4° C. and the serum was then isolated and stored at −20° C. until the analyses were performed. Each subject was followed to the end of gestation and pregnancy outcomes were collected and entered into a database. From this large cohort, 36 samples were retrieved from women known to have delivered a fetus with growth restriction (birth weight, 2451±80.9), defined as sex and gestational age adjusted birth weight less than the fifth centile compared to a Canadian reference population (19). None of these subjects was diagnosed with hypertension or preeclampsia in the index pregnancy. Thirty-six samples were retrieved from women who were healthy and who delivered normally grown fetuses (birth weight, 3501±43.4) at term. Neither the subjects nor the controls were smokers, used drugs or suffered medical complications. There were no fetal morphological or chromosomal abnormalities in either group.

Table 1 sets forth maternal demographic information in the study groups.

TABLE 1

| | Case (N = 36) | Normal (N = 36) | P value |
|---|---|---|---|
| Maternal Age (yrs) | 29.34 ± 0.78 | 31.11 ± 0.45 | P < 0.05 |
| Gravidity | 1.66 ± 0.16 | 2.17 ± 0.14 | P < 0.05 |
| Term deliveries | 0.182 ± 0.059 | 0.69 ± 0.09 | P < 0.001 |
| Preterm deliveries | 0.068 ± 0.038 | 0.034 ± 0.019 | 0.38 |
| Abortions | 0.40 ± 0.12 | 0.43 ± 0.09 | 0.81 |
| (spontaneous, | | | |
| ectopic, therapeutic) | | | |
| Living children | 0.16 ± 0.05 | 0.68 ± 0.08 | P < 0.0001 |
| History IUGR | 1 | 0 | |
| History | 2 | 3 | |
| Preeclampsia | | | |
| C sections (%) | 40% | 28% | 0.31* |
| Gestational age at | 38.68 ± 0.40 | 39.39 ± 0.13 | P < 0.05 |
| delivery (weeks) | | | |
| Birthweight (grams) | 2451 ± 80.9 | 3501 ± 43.4 | P < 0.0001 |
| Gestational age at | 12.65 ± 0.07 | 12.78 ± 0.05 | 0.16 |
| Sampling (weeks) | | | |

*chi-square test.
Others are student t-test

The maternal age, gravidity, number of term deliveries, number of living children, gestational age at delivery, and birth weight were all decreased in the study group (FGR) as compared to the control pregnancies (p<0.05). Forty percent of women in the study group were delivered by cesarian section compared with 26% in control pregnancies. There was no history of hypertension or preeclampsia in the index pregnancy in either group. Medication used by women in the study group included prometrium (1), flonase (1), and effexor (1). Medications in control pregnancies included acyclovir (1), rhinocort (1), calcium carbonate (1), nasonex (1), eltroxin (2), paxil (1), clonazepam (1), ventolin (1), synthroid (2), and progesterone (1). The data from each sample group was analyzed using student's T-test.

### Multi-Strip Western Blot

In order to exclude the variations caused by protein transfer, antibody incubation and exposure in different blots, multi-strip WB was conducted to assess all samples in the same blot, as described by others (27) with modification. For IGF-I, IGF-II, IGFBP-4 WB, serum samples were diluted (1:20) in 1× non-reducing protein loading buffer and boiled for 5 min.

Aliquots of 7.5 μl of diluted samples were applied to 15% tricine-SDS-PAGE with 15-well. Each gel contained 12 samples and protein ladder in both first and last well and as such, 6 gels were required to analyze all 72 samples. In addition, an internal control (pooled samples) was included on every gel. To assess IGFs, all six gels were excised under 17 kDa based on protein markers and aligned together to one nitrocellulose membrane for transfer. All gels were excised between 17 to 55 kDa and were also aligned and the proteins were transferred to another nitrocellulose membrane for IGFBP-4 determination. After transfer, the membranes were stained with Ponceau Red to confirm loading and transfer consistence. The membranes were then incubated with antibody extend solution (Thermo scientific, Rockford, Ill.) for 10 min at RT before blocking. After blocking, the membranes with transferred protein (under 17 kDa proteins) were incubated with IGF-I (#ab9572, abcam), or IGF-II antibodies (cloneS1F2, Upstate, Lake Placid, N.Y.) at 4° C. for overnight, respectively. IGFBP-4 was detected amongst the transferred proteins between 17-55 kDa by probing with IGFBP-4 antibody mentioned above. To assess PAPP-A, aliquots of 7.5 μl of diluted samples in 1× reducing loading buffer were applied to 7.5% tricine-SDS-PAGE with 15-well. All gels above 72 kDa were excised, aligned and transferred to one nitrocellulose membrane. This nitrocellulose membrane was probed with PAPP-A antibody (#AF2487, R&D systems). Band densities were quantified using Alpha Ease FCTM software.

### Statistical Analysis

Data quantified from multi-strip WB was expressed in median (25th, 75th percentiles) and presented as whisker box plots. Statistical significance was analyzed using Mann-Whitney U-test. Elevation of maternal circulating IGFBP-4 levels was defined when its level was greater than the 80th percentile in the nested control-case study. Odds ratio and their 95% confidence intervals were calculated with a standard 2×2 table. Statistical comparisons between circulating IGFBP-4 and PAPP-A between non-pregnant and pregnant women at different gestational ages were made by ANOVA, followed by Bonferroni post-hoc test. Linear correlation analysis of circulating IGFBP-4 and PAPP-A was also conducted. All statistical analysis were conducted by using Graph pad (prism 5) software. Statistical significance was set at p<0.05.

### B. Results

### IGFBP-4 and PAPP-A-1 are Highly Expressed by Decidual and Extravillous Trophoblasts at the Maternal-Fetal Interface

The expression of IGFBP-4 and PAPP-A-1 in the human placental bed in adjacent sections was examined. Cytokeratin 18 and vimentin were used as markers of extravillous trophoblasts and decidua, respectively.

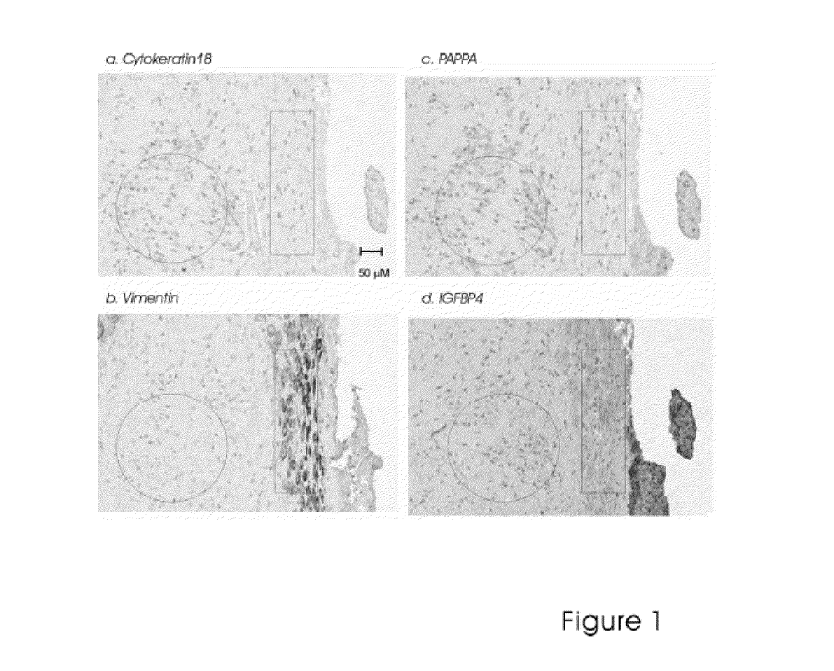

FIG. 1 depicts representative immunohistochemistry (IHC), revealing PAPP-A and IGFBP-4 expressed by decidual cells and extravillous trophoblasts in the maternal-fetal interface in early gestation. FIG. 1, panel a depicts areas of extravillous trophoblasts (i.e. cytokeratin positive) while FIG. 1, panel b depicts decidual cells (i.e. vimentin positive). Enrichment for each cell type is highlighted by a circle and box, respectively. FIG. 1, panels c and d show that both PAPP-A-1 and IGFBP-4 immunoreactivity was observed by both cell types. PAPP-A appeared to be highly expressed within the extravillous trophoblasts (as compared with decidual cells), while stronger immunoreactivity of IGFBP-4 was observed in decidual cells compared with those in extravillous trophoblasts.

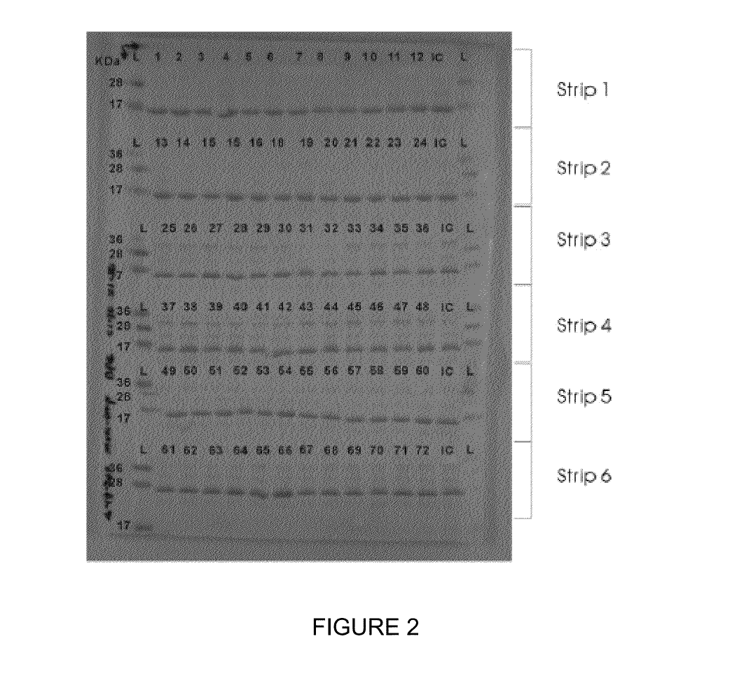

FIG. 2, panels a and b show that aliquots of 7.5 μl diluted serum samples (1:20) with 1× loading buffer as well as protein ladder and internal control were subjected to 6 SDS-PAGEs. Strips of gel in the range between 17 and 55 kDa were cut, aligned to a single membrane. The proteins in 6 strips of gel were transferred to a single membrane simultaneously. The membrane was stained with Ponceau Red solution. The even density of protein staining suggests equal sample loading and consistency of protein transfer.

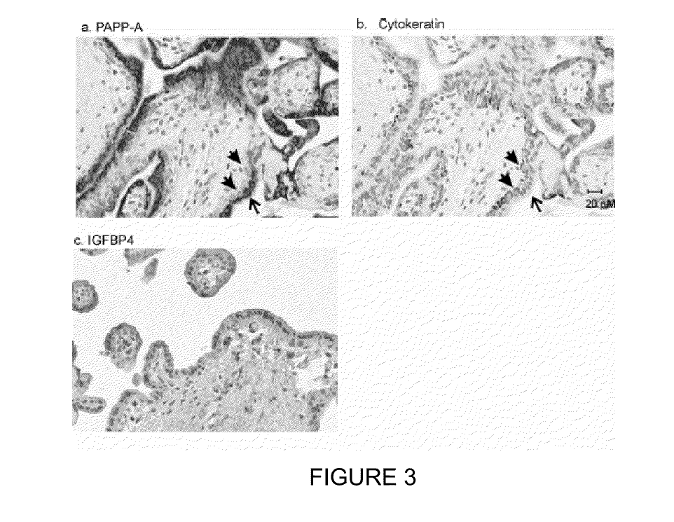

FIG. 3 depicts examination of PAPP-A-1 and IGFBP-4 expression within the villous region of the placenta, with strong PAPP-A-1 immunoreactivity observed within the syncytiotrophoblast layer (FIG. 3, panel a). PAPP-A is highly expressed by syncytiotrophoblasts in placental villi. A section containing placental villi at gestational age of 13 weeks was immunostained with PAPP-A (FIG. 3, panel a). After immunosignals were recorded, the section was destained and stain with cytokeratin (FIG. 3, panel b) as a cytotrophoblast trophoblast. Immunostaining of IGFBP-4 was also conducted in an adjacent section (FIG. 3, panel c). PAPP-A immunoactivity are strongly displayed by sycytiotrophoblasts (indicated by ←). Cytotrophoblast cells (indicated by [symbol] ) displaying cytokeratin positive signals were not stained by PAPP-A antibody. Modest IGFBP-4 immuno signals were displayed by cytotrophoblasts, syncytiotrophobalst and some villous stroma cells as well. The cytotrophoblasts, indicated by positive cytokeratin immunoreactivity, lacked PAPP-A-1 immuno cross-reactivity (FIG. 3, panel b). Only weak IGFBP-4 immunoreactivity was observed in the villous region of the placenta, with some staining observed in the cytotrophoblasts, syncytiotrophoblast and stromal cells (FIG. 3, panel c).

IGFBP-4 is the Predominant IGFBP Isoform Secreted by Human Extravillous Trophoblast, Capable of Forming a Complex with IGF-II

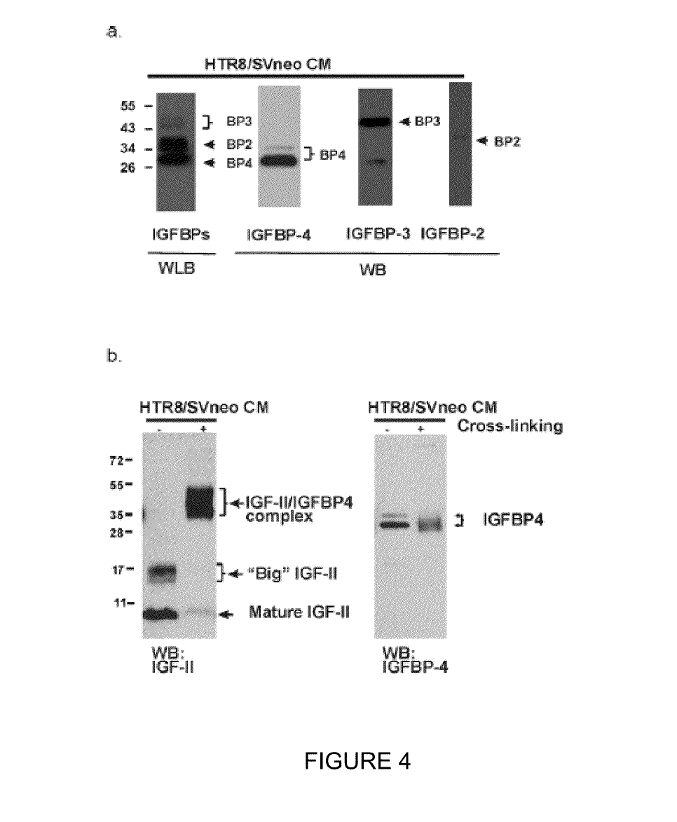

FIG. 4 depicts examination of secreted IGFBPs in the conditioned media of the HTR8/SVneo cells, which are representative of human extravillous trophoblast lineage. Concentrated spent media of HTR8/SVneo cells were subjected to WLB, and specific IGFBP detected by WLB was identified by WB; while WLB analysis of serum sample from non-pregnant women was used as a reference (FIG. 4, panel a). IGFBP-4 were found predominantly present in the conditioned medium, while IGFBP2 and IGFBP3 were also detected. IGFBP-4 are predominately secreted by human extravillous trophoblast cell line (HTR8/SVneo cells), and formed complex with IGF-II. FIG. 4, panel b shows the WB analysis of IGF-II and IGFBP-4 were conducted in conditioned media with or without cross-link. Mature IGF-II and “big” IGF-II were detected in conditioned media without cross-link, and dramatically decreased and appearance of larger molecular product cross-reacted with IGF-II antibody. IGFBP-4 WB analysis showed a decrease in IGFBP-4 in cross-linked samples. These data suggest IGFBP-4 is predominantly expressed by HTR8/SVneo cells and that it forms a complex with IGF-II. IGFBP2 (36 kDa), IGFBP3 (44, 46 kDa) and IGFBP-4 (28 kDa) were detected by WLB (FIG. 4, panel a). The specificity of each of these IGFBPs was confirmed by WB respectively. The predominant IGFBP secreted by this cell line was IGFBP-4 (FIG. 4, panel a). Mature and ‘big’ forms of IGF-II were also detected in conditioned medium by WB as shown at 9 kDa and 14-16 kDa, respectively (FIG. 4, panel b). Upon promotion of cross-linking, the molecular weight of almost all of the IGF protein bands were shifted to ˜40 kDa, paralleled by a significant reduction of the IGFBP-4 band at 28 kDa. These data suggest that IGFBP-4 is the predominant IGFBP secreted by HTR8/SVneo and that the majority of IGFBP-4 forms complexes with IGF-II.

IGFBP-4 Protein Expression is Increased in Early Pregnancy, while PAPP-A Content Increases as Gestation Progresses, in the Maternal Circulation.

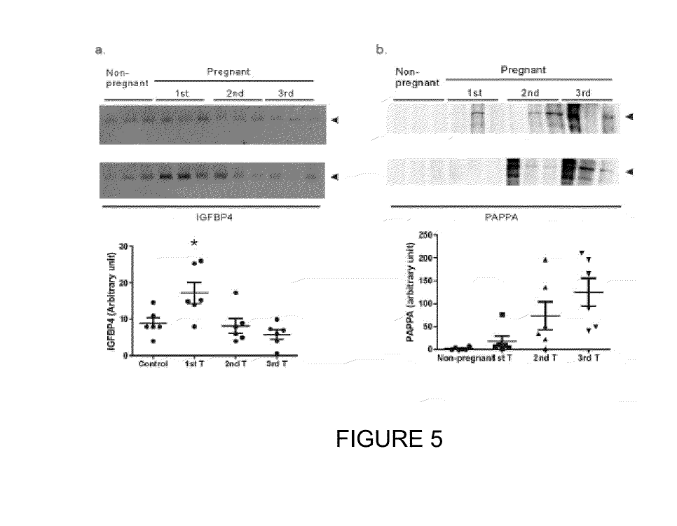

FIG. 5 depicts a gestational profile of circulating concentrations of IGFBP-4 and PAPP-A, as assessed in healthy non-pregnant and pregnant women by WB analysis. Circulating IGFBP-4 protein content was significantly higher in 1st trimester serum samples compared to samples of non-pregnant women and women in their 2nd and 3rd trimester of pregnancy (FIG. 5, panel a). PAPP-A was occasionally detected as early as 6 weeks of pregnancy in the maternal circulation, and increased with advancing gestation (FIG. 5, panel b). Therefore, IGFBP-4 is increased in maternal circulation in pregnant women at early gestational age, while PAPP-A increases as gestation progresses. This has not been demonstrated previously.

Maternal Levels of IGF-I and IGF-II During the First Trimester are not Associated with the Development of IUGR

To examine whether circulating maternal levels of IGF-I or IGF-II in the first trimester were correlated with the subsequent development of IUGR, a nested control-case study was conducted. Circulating IGF-I and II concentrations were determined with multi-strip WB method. Consistent loading and transfer efficiency was confirmed by showing an even protein density in blot with Ponceau Red staining (FIG. 2).

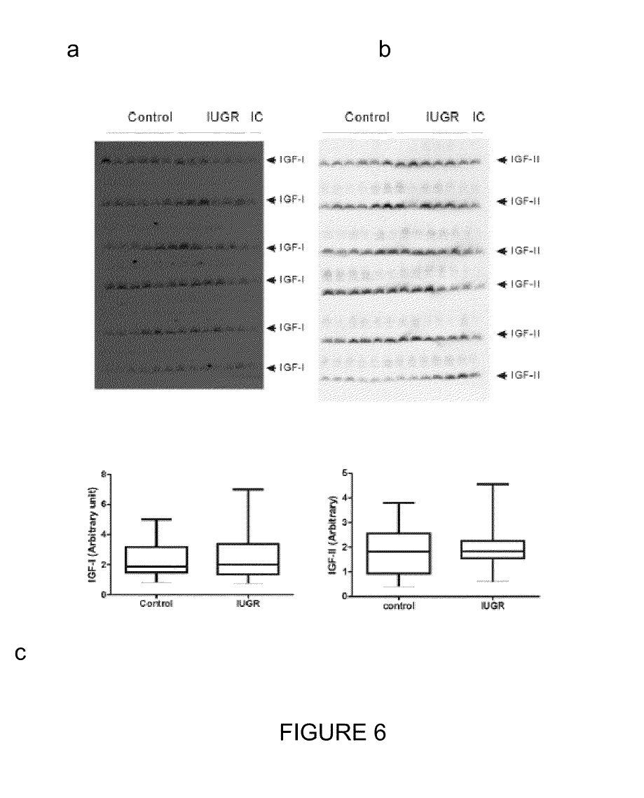

FIG. 6 shows there was no difference in either IGF-1 or IGF-II levels between pregnant women destined to deliver an IUGR fetus and those delivering an infant of average weight for gestation.

Elevation of Circulating Maternal IGFBP-4 Levels in Early Gestation is Associated with the Development of IUGR

FIG. 7 depicts the results of experiments which measured circulating IGFBP-4 protein levels in 1st trimester serum samples from the same two groups of women. FIG. 7, panel a depicts a multi-strip WB showing that IGFBP-4 protein content was significantly higher in women who went on to deliver a growth restricted baby (birth weight<5th centile) compared with the IGFBP-4 content (0.48; 0.28-0.74) in those delivering a normal size infant. Circulating IGFBP-4 levels are elevated in the women who were destined to deliver IUGR baby at early gestational age (FIG. 7, panels a and b). This has not been shown previously.

Table 2 depicts elevated (+) vs. non-elevated (−) circulating IGFBP-4 in pregnant women who went on to deliver a growth restricted (IUGR+) vs. normal (IUGR−) birth weight baby.

TABLE 2

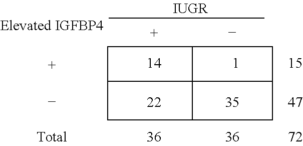

Fourteen cases in the IUGR group (top left quadrant of Table 2) and 1 control (top right quadrant) were found to have elevated IGFBP-4 levels as defined as larger than eightieth percentiles. Thus, the odds ratio for the development of IUGR with elevated IGFBP-4 in early pregnancy was 22.3 (95% confidence interval=2.7-181.5), and sensitivity 38.8%, specificity 97.2%, positive predictive value 93.3% and negative predictive value 74.4% (Table 2). No difference was found in circulating PAPP-A protein content between the two groups of women (1.39; 0.80-2.55 in control vs. 1.42; 0.65-2.30 in FGR) (FIG. 7, panel b). Linear regression analysis indicated no correlation between circulating IGFBP-4 and PAPP-A in either group.

### C. Discussion

In Example 1, the strong association between the elevation of IGFBP-4 levels in the maternal circulation in the first trimester and the later development of IUGR has been demonstrated. In contrast, all other components of the IGF system assessed here were similar between women destined to develop an IUGR fetus and healthy controls. This is the first documented study showing that circulating maternal IGFBP-4 levels measured during the first trimester of pregnancy can be used as a predictor of pregnant women who are destined to deliver an IUGR baby.

Furthermore, the data in Example 1 suggest that IGFBP-4 is a key regulator of IGF-II mediated placenta and fetal development. The increase in IGFBP-4 levels in the maternal circulation coincides with the time during which the population of cells that expresses IGFBP-4 is most abundant in the placental bed. On the contrary, circulating PAPP-A levels increase with advancing gestation which is consistent with the notion that PAPP-A is mostly expressed by the syncytiotrophoblast. No correlation between circulating PAPP-A and IGFBP-4 was observed.

Increased levels of IGFBP-4 in the maternal circulation in early pregnancy, paralleled by an abundance of cells (maternal decidual cells and extravillous trophoblasts) with high expression of IGFBP-4 at this time (FIG. 1) suggests that circulating levels of IGFBP-4 may originate from the maternal-fetal interface and reflect an abundance of this protein in this microenvironment. A likely explanation for elevated IGFBP-4 is that deficiencies in IGFBP-4 proteolysis by PAPP-A, also shown to be localized at the maternal-fetal interface. This is supported by increased IGFBP-4 levels found in the circulation of PAPP-A null mice (9). Alternatively, an increase in circulating IGFBP-4 may be simply due to overexpression of this protein in the tissue.

A significant elevation in circulating IGFBP-4 in the first trimester (11-13 weeks) was associated with the later development of IUGR. The timing of such events is such that the elevation of IGFBP-4 in the placental bed could represent a cause of placental dysfunction, rather than being merely the consequence of impaired placental development, as supported by studies of PAPP-A and PAPP-A/IGFBP-4 double knockout mice. Indeed, PAPP-A knock-out mice fail to process IGFBP-4, resulting in an increase in circulating maternal IGFBP-4 levels and a 34% growth deficiency compared to wild type litter mates (8). The growth restriction observed in the PAPP-A knock out mouse is however attenuated when the mouse is additionally null for IGFBP-4 (9), suggesting increased IGFBP-4 in PAPP-A knockout mouse contributes significantly to fetal growth restriction. However, in the PAPP-A knockout mouse study, the levels of IGFBP-4 were not measured and directly linked to IUGR. Rather, it was found that PAPP-A is an essential growth regulatory factor in vivo, and that a novel mechanism for regulated IGF bioavailability during early fetal development could be suggested.

A certain amount of IGFBP-4 is likely required in the placental bed for IGF-II to maintain a longer half-life, by forming an IGFBP-4/IGF-II complex. Whenever tissues require IGF-II's action, PAPP-A processing of IGFBP-4 from the IGFBP-4/IGF-II complex frees IGF-II. It has been suggested that IGFBP-4 may thus serve as a reservoir of IGF-II within the placental bed, positively regulating the bioavailability of IGF-II (9).

Immunohistochemsitry data clearly demonstrated that PAPP-A is present at the maternal-fetal interface, and is expressed by extravillous trophoblasts and maternal decidual cells (FIG. 3). This is consistent with the detection of PAPP-A in the conditioned medium of extravillous trophoblasts and that in decidualized endometrial stromal cells (7). Due to the co-localized expression of IGFBP-4 at this interface, this site of PAPP-A expression highlights PAPP-A's proteolytic processing function of IGFBP-4 at the maternal-fetal interface. Within the villous portion of the placenta PAPP-A is exclusively expressed by syncytiotrophoblast which is directly in contact with the maternal circulation. This site of PAPP-A production is likely the primary source of this protease within the maternal circulation. Although decidual endometrial stromal cells and extravillous trophoblasts are more abundant in the 1st trimester, circulating PAPP-A levels appear relatively low (FIG. 5, panel b), suggesting that PAPP-A expressed at the maternal-fetal interface makes little contribution to circulating levels of IGFBP-4. On the other hand, the syncytial surface increases significantly as gestation progresses, consistent with observations of increased circulating PAPP-A from 1st to 3rd trimester. This is also consistent with in vitro data showing significant increases in PAPP-A mRNA following syncytialization of cultured villous cytotrophoblasts (21). This observation may also explain the reduced maternal PAPP-A concentrations found in women carrying fetuses with trisomy 21. Defective syncytialization has been demonstrated both in vivo (22) and in vitro (23; 24) in placentae obtained from affected fetuses. A lack of correlation between circulating PAPP-A and IGFBP-4 levels (FIG. 7) is supported in Example 1, suggesting circulating PAPP-A concentrations may not accurately reflect the proteolytic processing of IGFBP-4 and/or the bioavailability of IGFs at the maternal-fetal interface. Indeed, 99% of PAPP-A in the circulation is present as a heterotetrameric complex with 2 subunits of PAPP-A and 2 subunits of proMBP (25), an inhibitor of the proteolytic activity of PAPP-A (26). The subset of women with lower circulating PAPP-A have a significantly increased risk of IUGR, extreme preterm delivery, preeclampsia and stillbirth (27-29). However, the lower PAPP-A in the circulation is not necessarily the consequence of defective syncytialization during placental development, rather than the cause of inadequate syncytiotrophoblast formation. In the results presented above, there was no association between circulating PAPP-A (first trimester) and the development of IUGR.

By using multi-strip WB, it has been demonstrated for the first time that maternal IGFBP-4 in early gestation is associated with an increased risk of delivering a FGR fetus. This is supported by the data on IGFBP-4 inhibiting IGF-II activity as an underling mechanism in the development of IUGR. In the study above, the odds ratio for the development of IUGR, given an abnormally high IGFBP-4 in early pregnancy reached 22(95% Cl 2.7-181), which is much higher than any reported biomarker alone including PAPP-A (3.9 (95% Cl 2.3-6.5)(16; 31; 35), free βHCG (2.7, 95% Cl 1.3-5.9) (31), and AFP 5.7 (95% Cl 2.7-12.7) (32). Given the high positive predictive value observed, IGFBP-4 is a useful predictor of pregnancies destined to be complicated by IUGR. This is clinically relevant as it should permit the targeting of pregnancies which warrant closer follow-up and possible therapeutic interventions for IUGR. Since there are practical limitations with using a multi-strip WB as an assay for determining maternal serum IGFBP-4 levels, another type of quantitative assay can be used. For example, an ELISA would satisfy the practical requirements of testing for the presence and levels of IGFBP-4 in the maternal serum.

Indeed, circulating IGFs and IGFBPs have been extensively examined in IUGR by using ELISAs (36-41). IGFBP-4 levels were in serum samples with a standard, commercially available ELISA Kit (Cat #DY804, R&D system), however the data was not consistent with multi-WB results (data not shown). IGFBP-4 concentrations as determined by this particular ELISA Kit were significantly underestimated in samples in which recombinant IGF-II were added (data not shown), suggesting that IGFBP-4 determination in this particular ELISA is interfered with by the presence of IGFs in the samples. This could readily be overcome by devising an ELISA using another antibody whose binding is subject to interference by IGFBP-4/IGF complexation, or through disruption of complexes (see below).

On the other hand, conventional WB is able to separate different isoforms of a protein according to molecular size. Multi-strip WB methodology may be applied (as above) to assess all samples (72 cases) simultaneously in one single blot, thereby avoiding variations in protein transfer, antibody incubation and conditions in visualization of signals. The results are objective and each individual sample result is presented in one blot.

In summary, it has been demonstrated in that elevated IGFBP-4 levels in the maternal circulation during early pregnancy are highly associated with subsequent development of IUGR. IGFBP-4 is most likely a key regulator of the IGF-II system, tightly involved in regulating the bioavailability of IGFs, and thereby playing an important role in modulating placental and fetal growth. The elevation of circulating IGFBP-4 in early pregnancy may reflect an abnormally high protein content of IGFBP-4 in the placental bed, subsequently reducing IGF-II bio-availability, thus leading to impaired placental development and fetal growth restriction. Therefore, above findings provide support for the clinical use of IGFBP-4 as an early biomarker for IUGR.

### Example 2

### Detection of IGFBP-4 by ELISA

As described above, it is of clinical relevance to have a simple test to detect the levels of IGFBP-4 in maternal serum levels during the first trimester of pregnancy.

On the basis of the data presented herein, an ELISA could be developed which would measure IGFBP-4, and would detect the amounts of this protein normally bound to IGF-II, thus giving us a better estimate of the amount of the protein present.

This could be accomplished through development of an antibody which binds to IGFBP-4 in a manner which is not limited by complexation of IGFBP-4 with IGF-II or other proteins. For instance, a suitable antibody could bind to an epitope in IGFBP-4 which is not sterically hindered or otherwise obstructed by binding of IGFBP-4 to IGF-II or other proteins.

Alternatively, interfering complexes could be disrupted prior to IGFBP-4 detection. For example, the IGF-II/IGFPBP-4 complex could be disrupted prior to measuring levels of IGFBP-4 in serum. One means of achieving this would be through addition an inhibitor which disrupts or promotes dissociation of complexes comprising IGFBP-4, including IGFBP-4/IGF-II complexes. Such inhibitors could be competitive or non-competitive. An example of a competitive inhibitor is a molecule which binds to IGF-II with higher affinity than IGFBP-4. An example of one such inhibitor is IGFBP-3. Preliminary experiments (not shown) indicates that addition IGFBP-3 to a sample (e.g. a serum sample) is effective for disrupting IGF-II/IGFPBP-4 complexes and improving detection of IGFBP-4 in the ELISA.

### REFERENCES

1. Hack M, Merkatz I R 1995 Preterm delivery and low birth weight—a dire legacy. N Engl J Med 333:1772-1774
2. Kaufmann P, Black S, Huppertz B 2003 Endovascular trophoblast invasion: implications for the pathogenesis of intrauterine growth retardation and preeclampsia. Biol Reprod 69:1-7
3. Fowden A L 2003 The insulin-like growth factors and feto-placental growth. Placenta 24:803-812
4. Baker J, Liu J P, Robertson E J, Efstratiadis A 1993 Role of insulin-like growth factors in embryonic and postnatal growth. Cell 75:73-82
5. Han V K, Bassett N, Walton J, Challis J R 1996 The expression of insulin-like growth factor (IGF) and IGF-binding protein (IGFBP) genes in the human placenta and membranes: evidence for IGF-IGFBP interactions at the feto-maternal interface. J Clin Endocrinol Metab 81:2680-2693
6. Lawrence J B, Oxvig C, Overgaard M T, Sottrup-Jensen L, Gleich G J, Hays L G, Yates J R, III, Conover C A 1999 The insulin-like growth factor (IGF)-dependent IGF binding protein-4 protease secreted by human fibroblasts is pregnancy-associated plasma protein-A. Proc Natl Acad Sci USA 96:3149-3153
7. Giudice L C, Conover C A, Bale L, Faessen G H, Ilg K, Sun I, Imani B, Suen L F, Irwin J C, Christiansen M, Overgaard M T, Oxvig C 2002 Identification and regulation of the IGFBP-4 protease and its physiological inhibitor in human trophoblasts and endometrial stroma: evidence for paracrine regulation of IGF-II bioavailability in the placental bed during human implantation. J Clin Endocrinol Metab 87:2359-2366
8. Conover C A, Bale L K, Overgaard M T, Johnstone E W, Laursen U H, Fuchtbauer E M, Oxvig C, van Deursen J 2004 Metalloproteinase pregnancy-associated plasma protein A is a critical growth regulatory factor during fetal development. Development 131:1187-1194
9. Ning Y, Schuller A G, Conover C A, Pintar J E 2008 Insulin-like growth factor (IGF) binding protein-4 is both a positive and negative regulator of IGF activity in vivo. Mol Endocrinol 22:1213-1225
10. Brambati B, Macintosh M C, Teisner B, Maguiness S, Shrimanker K, Lanzani A, Bonacchi I, Tului L, Chard T, Grudzinskas J G 1993 Low maternal serum levels of pregnancy associated plasma protein A (PAPP-A) in the first trimester in association with abnormal fetal karyotype. Br J Obstet Gynaecol 100:324-326
11. Nicolaides K H 2011 Screening for fetal aneuploidies at 11 to 13 weeks. Prenat Diagn 31:7-15
12. Wright D, Spencer K, Kagan K K, Torring N, Petersen O B, Christou A, Kallikas J, Nicolaides K H 2010 First-trimester combined screening for trisomy 21 at 7-14 weeks' gestation. Ultrasound Obstet Gynecol 36:404-411
13. Dugoff L, Hobbins J C, Malone F D, Porter T F, Luthy D, Comstock C H, Hankins G, Berkowitz R L, Merkatz I, Craigo S D, Timor-Tritsch I E, Carr S R, Wolfe H M, Vidaver J, D'Alton M E 2004 First-trimester maternal serum PAPP-A and free-beta subunit human chorionic gonadotropin concentrations and nuchal translucency are associated with obstetric complications: a population-based screening study (the FASTER Trial). Am J Obstet Gynecol 191:1446-1451
14. Goetzl L, Krantz D, Simpson J L, Silver R K, Zachary J M, Pergament E, Platt L D, Mahoney M J, Wapner R J 2004 Pregnancy-associated plasma protein A, free beta-hCG, nuchal translucency, and risk of pregnancy loss. Obstet Gynecol 104:30-1
15. Spencer K, Cowans N J, Molina F, Kagan K O, Nicolaides K H 2008 First-trimester ultrasound and biochemical markers of aneuploidy and the prediction of preterm or early preterm delivery. Ultrasound Obstet Gynecol 31:147-152
16. Smith G C, Stenhouse E J, Crossley J A, Aitken D A, Cameron A D, Connor J M 2002 Early pregnancy levels of pregnancy-associated plasma protein a and the risk of intrauterine growth restriction, premature birth, preeclampsia, and stillbirth. J Clin Endocrinol Metab 87:1762-1767
17. Spencer K, Cowans N J, Avgidou K, Nicolaides K H 2006 First-trimester ultrasound and biochemical markers of aneuploidy and the prediction of impending fetal death. Ultrasound Obstet Gynecol 28:637-643
18. Qiu Q, Yan X, Bell M, Di J, Tsang B K, Gruslin A 2010 Mature IGF-II prevents the formation of “big” IGF-II/IGFBP-2 complex in the human circulation. Growth Horm IGF Res 20:110-117
19. Wen S W, Fung K F, Huang L, Demissie K, Joseph K S, Allen A C, Kramer M S 2005 Fetal and neonatal mortality among twin gestations in a Canadian population: the effect of intrapair birthweight discordance. Am J Perinatol 22:279-286
20. Kiyatkin A, Aksamitiene E 2009 Multistrip western blotting to increase quantitative data output. Methods Mol Biol 536:149-161
21. Guibourdenche J, Frendo J L, Pidoux G, Bertin G, Luton D, Muller F, Porquet D, Evain-Brion D 2003 Expression of pregnancy-associated plasma protein-A (PAPP-A) during human villous trophoblast differentiation in vitro. Placenta 24:532-539
22. Roberts L, Sebire N J, Fowler D, Nicolaides K H 2000 Histomorphological features of chorionic villi at 10-14 weeks of gestation in trisomic and chromosomally normal pregnancies. Placenta 21:678-683
23. Pidoux G, Gerbaud P, Marpeau O, Guibourdenche J, Ferreira F, Badet J, Evain-Brion D, Frendo J L 2007 Human placental development is impaired by abnormal human chorionic gonadotropin signaling in trisomy 21 pregnancies. Endocrinology 148:5403-5413
24. Massin N, Frendo J L, Guibourdenche J, Luton D, Giovangrandi Y, Muller F, Vidaud M, Evain-Brion D 2001 Defect of syncytiotrophoblast formation and human chorionic gonadotropin expression in Down's syndrome. Placenta 22 Suppl A:S93-597
25. Oxvig C, Sand O, Kristensen T, Gleich G J, Sottrup-Jensen L 1993 Circulating human pregnancy-associated plasma protein-A is disulfide-bridged to the proform of eosinophil major basic protein. J Biol Chem 268:12243-12246
26. Soe R, Overgaard M T, Thomsen A R, Laursen L S, Olsen I M, Sottrup-Jensen L, Haaning J, Giudice L C, Conover C A, Oxvig C 2002 Expression of recombinant murine pregnancy-associated plasma protein-A (PAPP-A) and a novel variant (PAPP-Ai) with differential proteolytic activity. Eur J Biochem 269:2247-2256
27. Leung T Y, Sahota D S, Chan L W, Law L W, Fung T Y, Leung T N, Lau T K 2008 Prediction of birth weight by fetal crown-rump length and maternal serum levels of pregnancy-associated plasma protein-A in the first trimester. Ultrasound Obstet Gynecol 31:10-14
28. Goetzinger K R, Singla A, Gerkowicz S, Dicke J M, Gray D L, Odibo A O 2009 The efficiency of first-trimester serum analytes and maternal characteristics in predicting fetal growth disorders. Am J Obstet Gynecol 201:412-416
29. Ong C Y, Liao A W, Spencer K, Munim S, Nicolaides K H 2000 First trimester maternal serum free beta human chorionic gonadotrophin and pregnancy associated plasma protein A as predictors of pregnancy complications. BJOG 107:1265-1270
30. Morssink L P, Kornman L H, Hallahan T W, Kloosterman M D, Beekhuis J R, de Wolf B T, Mantingh A 1998 Maternal serum levels of free beta-hCG and PAPP-A in the first trimester of pregnancy are not associated with subsequent fetal growth retardation or preterm delivery. Prenat Diagn 18:147-152
31. Krantz D, Goetzl L, Simpson J L, Thom E, Zachary J, Hallahan T W, Silver R, Pergament E, Platt L D, Filkins K, Johnson A, Mahoney M, Hogge W A, Wilson R D, Mohide P, Hershey D, Wapner R 2004 Association of extreme first-trimester free human chorionic gonadotropin-beta, pregnancy-associated plasma protein A, and nuchal translucency with intrauterine growth restriction and other adverse pregnancy outcomes. Am J Obstet Gynecol 191:1452-1458
32. Kirkegaard I, Henriksen T B, Uldbjerg N 2011 Early fetal growth, PAPP-A and free beta-hCG in relation to risk of delivering a small-for-gestational age infant. Ultrasound Obstet Gynecol 37:341-347
33. Proctor L K, Toal M, Keating S, Chitayat D, Okun N, Windrim R C, Smith G C, Kingdom J C 2009 Placental size and the prediction of severe early-onset intrauterine growth restriction in women with low pregnancy-associated plasma protein-A. Ultrasound Obstet Gynecol 34:274-282
34. Pilalis A, Souka A P, Antsaklis P, Daskalakis G, Papantoniou N, Mesogitis S, Antsaklis A 2007 Screening for pre-eclampsia and fetal growth restriction by uterine artery Doppler and PAPP-A at 11-14 weeks' gestation. Ultrasound Obstet Gynecol 29:135-140
35. Montanan L, Alfei A, Albonico G, Moratti R, Arossa A, Beneventi F, Spinillo A 2009 The impact of first-trimester serum free beta-human chorionic gonadotropin and pregnancy-associated plasma protein A on the diagnosis of fetal growth restriction and small for gestational age infant. Fetal Diagn Ther 25:130-135
36. Baker M, Lopes Moreira M E, Sichieri R, Moura A S 2009 Reduction of IGF-binding protein-3 as a potential marker of intra-uterine growth restriction. J Perinat Med 37:689-693
37. Burkhardt T, Matter C M, Lohmann C, Cai H, Luscher T F, Zisch A H, Beinder E 2009 Decreased umbilical artery compliance and igf-I plasma levels in infants with intrauterine growth restriction—implications for fetal programming of hypertension. Placenta 30:136-141
38. Davidson S, Hod M, Merlob P, Shtaif B 2006 Leptin, insulin, insulin-like growth factors and their binding proteins in cord serum: insight into fetal growth and discordancy. Clin Endocrinol (Oxf) 65:586-592
39. Bocconi L, Mauro F, Maddalena S E, De I C, Tirelli A S, Pace E, Nicolini U 1998 Insulinlike growth factor 1 in controls and growth-retarded fetuses. Fetal Diagn Ther 13:192-196
40. Hills F A, English J, Chard T 1996 Circulating levels of IGF-I and IGF-binding protein-1 throughout pregnancy: relation to birthweight and maternal weight. J Endocrinol 148:303-309
41. Klauwer D, Blum W F, Hanitsch S, Rascher W, Lee P D, Kiess W 1997 IGF-I, IGF-II, free IGF-I and IGFBP-1, -2 and -3 levels in venous cord blood: relationship to birthweight, length and gestational age in healthy newborns. Acta Paediatr 86:826-833
42. Gorecki D C, Beresewicz M, Zablocka B 2007 Neuroprotective effects of short peptides derived from the Insulin-like growth factor 1. Neurochem Int 51:451-458
43. Qiu Q, Basak A, Mbikay M, Tsang B, Gruslin A 2005 Role of prolGF-11 processing by proprotein convertase 4 in human placental development. Proc Natl Acad Sci USA 102:11047-11052
44. Mazerbourg S, Callebaut I, Zapf J, Mohan S, Overgaard M, Monget P 2004 Up date on IGFBP-4: regulation of IGFBP-4 levels and functions, in vitro and in vivo. Growth Horm IGF Res 14:71-84
45. Zhong, Y., Tuuli, M., Odibo, A. O. 2010 First-trimester assessment of placenta function and the prediction of preeclampsia and intrauterine growth restriction. Prenat Diagn 2010; 30: 293-308
46. Brask, D., Høgdall, E., Johansen, J. and Skibsted, L. 2009 OC29.02: Serum YKL-40—a potential new biomarker for preeclampsia and intrauterine growth restriction. Ultrasound in Obstetrics & Gynecology 2009; 34 (Suppl. 1): p. 56.
47. Chafetz, I., Kuhnreich, I., Sammar, M. Tai, Y., Gibor, Y., Meiri, H., Cuckle, H. and Wolf, M. 2007 First-trimester placental protein 13 screening for preeclampsia and intrauterine growth restriction American Journal of Obstetrics and Gynecology, Volume 197, Issue 1, Pages 35.e1-35.e7
48. Barekhell-Thomas, A., Tong, S., Baker, L. S., Edwards, A. and Wallace, E. M. Maternal serum activin A and the prediction of intrauterine growth restriction. 2006. Australian and New Zealand Journal of Obstetrics and Gynaecology, 46: 97-101)
49. Srinivas, S. K., Edlow, A. G., Neff, P. M., Samuel, M. D., Andrela, C. M. and Elovitz, M. A. Rethinking IUGR in preeclampsia: dependent or independent of maternal hypertension? 2009. J. Perinatol. 29(10): 680-684.
50. Mark C. Walker, Sara A. Finkelstein, Ruth Rennicks White, Svetlana Shachkina, Graeme N. Smith, Shi Wu Wen, Marc Rodger 2011 The Ottawa and Kingston (OaK) Birth Cohort: Development and Achievements.
51. Tapanainen et al. Maternal hypoxia as a model for intrauterine growth retardation: effects on insulin-like growth factors and their binding proteins. Pediatr Res. 1994 August; 36(2):152-8.
52. Carter et al. Altered expression of IGFs and IGF-binding proteins during intrauterine growth restriction in guinea pigs. J. Endocrinol. 2005 January; 184(1):179-89.
53. de Vrijer B. et al. Altered placental and fetal expression of IGFs and IGF-binding proteins associated with intrauterine growth restriction in fetal sheep during early and mid-pregnancy. Pediatr Res. 2006 November; 60(5):507-12.
54. U.S. Patent Publication No. 2010/0016173.
55. Am J. Pathol. 2010 December; 177(6):2950-62. Epub 2010 Oct. 15.
56. Placenta. 2009 March; 30(S): 43-48.
57. Placenta. 2008 June; 29(6): 555-563.
58. Morrison et al., Proc. Natl. Acad. Sci. U.S.A. 81, 6851 (1985).
59. Takeda et al., Nature 314, 452 (1985).
60. Cabilly et al., U.S. Pat. No. 4,816,567.
61. Boss et al., U.S. Pat. No. 4,816,397.
62. European Patent Publication EP171496.
63. European Patent Publication 0173494.
64. United Kingdom Patent GB 2177096B.
65. Teng et al, Proc. Natl. Acad. Sci. U.S.A., 80, 7308-7312 (1983)
66. Kozbor et al., Immunology Today, 4, 7279 (1983)
67. Olsson et al., Meth. Enzymol., 92, 3-16 (1982)
68. PCT Publication WO92/06193
69. European Patent Document EP0239400
70. Roberts et al. Placenta, Volume 23, Issue 10, November 2002, Pages 763-770.
71. U.S. Pat. No. 7,790,463.
72. U.S. Patent Publication No. 2007/017605.
73. Cooper et al. Prenat Diagn. 2009 March; 29(3):248-52.
74. Christians and Gruslin Prenat Diagn 2010; 30: 815-820.
75. U.S. Pat. No. 7,955,805.
76. U.S. Patent Publication No. 2010/0016173.
77. U.S. Patent Publication No. 2010/0304412.
78. Spencer et al. Prenat Diagn 2005; 25: 949-953.
79. Pihl et al. Prenat Diagn 2008; 28: 1131-1135.

All references cited herein are incorporated by reference in their entirety.

In the preceding description, for purposes of explanation, numerous details are set forth in order to provide a thorough understanding of the embodiments. However, it will be apparent to one skilled in the art that these specific details are not required. The above-described embodiments are intended to be examples only. Alterations, modifications, and variations can be effected to the particular embodiments by those of skill in the art without departing from the scope, which is defined solely by the claims appended hereto.

What is claimed is:

1. A method of determining risk of intrauterine growth restriction (IUGR) during pregnancy comprising the steps of:
obtaining a sample from a pregnant woman,
taking a measurement indicative of an IGFBP-4 level in the sample, and
determining that an increased risk of IUGR exists if the IGFBP-4 level is elevated.

2. The method of claim 1, wherein the sample comprises blood, serum, plasma, urine, or amniotic fluid.

3. The method of claim 2, wherein the sample comprises serum.

4. The method of claim 1, wherein the sample is obtained during the first trimester of pregnancy.

5. The method of claim 1, wherein the pregnant woman is a woman having a personal or family history of:
IUGR;
pre-eclampsia;
eclampsia;
HELLP syndrome;
hypertension;
vascular disease;
auto-immune disease;
renal disease; or
diabetes.

6. The method of claim 1, wherein the measurement is a measurement of IGFBP-4 protein, IGFBP-4 mRNA, or a genetic variant of IGFBP-4 which leads to increased expression or activity.

7. The method of claim 6, wherein the measurement is a measurement of IGFBP-4 protein.

8. The method of claim 7 wherein, prior to the step of taking a measurement, an inhibitor is added to disrupt complexes comprising IGFBP-4.

9. The method of claim 8, wherein the complexes are complexes comprising IGFBP-4 and IGF-II.

10. The method of claim 9, wherein the inhibitor is a competitive inhibitor.

11. The method of claim 10, wherein the competitive inhibitor comprises IGFBP-3.

12. The method of claim 1, wherein the IGFBP-4 level is considered to be elevated if it is higher than eightieth percentile.

13. The method of claim 12, wherein the sensitivity of the method is about 38% or the specificity is about 97%.

14. The method of claim 13, wherein the positive predictive value of the method is about 93% or the negative predictive value is about 74%.

15. A kit for determining a risk of intrauterine growth restriction (IUGR) during pregnancy from a sample obtained from a pregnant woman comprising:
reagents for taking a measurement indicative of an IGFBP-4 level in said sample; and
instructions for use,
wherein an increased risk of IUGR is determined if the level of IGFBP-4 is elevated.

16. The kit of claim 15, wherein the sample comprises blood, serum, plasma, urine, or amniotic fluid.

17. The kit of claim 16, where the sample comprises serum.

18. The kit of claim 15, wherein the kit is for determining the risk of intrauterine growth restriction during the first trimester of pregnancy.

19. The kit of claim 15, wherein the sample is from a pregnant woman having a personal or family history of:
IUGR;
pre-eclampsia;
eclampsia;
HELLP syndrome;
hypertension;
vascular disease;
auto-immune disease;
renal disease; or
diabetes.

20. The kit of claim 15, wherein the reagents are for measuring IGFBP-4 protein, IGFBP-4 mRNA, or a genetic variant of IGFBP-4 which leads to increased expression or activity.
